(19) United States
(12) Patent Application Publication
(10) Pub. No.: US 2011/0269790 A1
(43) Pub. Date: Nov. 3, 2011

(54) NOVEL THIENOPYRIDINES AS PHARMACOLOGICALLY ACTIVE AGENTS

(76) Inventors: Shireesha Boyapati, Warangal (IN); Akkinepally Raghuram Rao, Chandigarh (IN); Banda Narsaiah, Hyderabad (IN); Prakash V. Diwan, Hyderabad (IN); Sistla Ramakrishna, Hyderabad (IN); Madhusudhana, Hyderabad (IN); Tallapally Yakaiah, Hyderabad (IN)

(21) Appl. No.: 13/143,272

(22) PCT Filed: Jan. 5, 2010
(86) PCT No.: PCT/IN2010/000005

§ 371 Date: Jul. 5, 2011

(30) Foreign Application Priority Data
Jan. 5, 2009 (IN) 10/DEL/2009

(51) Int. Cl. A61K 31/4365; A61P 19/00; A61P 35/00; C07D 409/02; A61P 3/10

(52) U.S. Cl. 514/285; 546/80; 546/62; 514/291

(57) ABSTRACT

The present invention provides compounds and pharmaceutically acceptable salts thereof methods for synthesizing thienopyridines and methods for inhibiting TNF-α activity for the treatment of cancer, asthma, arthritis, diabetes and inflammation. Provided are compounds of formula (I).

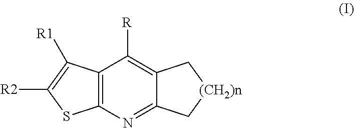

### FIELD OF THE INVENTION

The present invention relates to novel thienopyridines and to pharmaceutically acceptable salts thereof, that are useful as pharmacologically active agents, and to prodrugs thereof. The present invention also provides methods for the synthesis of such compounds and to new and/or improved anti-inflammatory, anti-arthritic, anti-cancer, anti-diabetic and adenoside antagonistic activity of such compounds and/or their salts.

### BACKGROUND OF THE INVENTION

The benzene/thiophene nucleus represents one of the most prominent examples of bioisosterism. The specific placement of a thiophene moiety is considered a promising area of research and is a potential area of choice in drug design and is believed to be superior to the use of benzene due to its lipophilicity.

Synthesis of polysubstituted thiophenes from multicomponent condensation of ketones or aldehydes, cyanoacetate and elemental sulfur was originally published in 1961 by Gewald et al. Several patents describe the synthesis and technical importance of thiophene as disperse dyes and their application on synthetic fibers. Dyes derived from thiophene have many advantages such as the colour deepening effect as an intrinsic property of thiophene ring. Its' small molecular structure also leads to better dye ability. Beyond their industrial use as dyes and conducting polymers, highly substituted thiophenes have shown extensive potential in pharmaceutical industry.

Other than the above, some derivatives of thiophenes are reported as analgesic, antiinflammatory, anticancer and CNS active agents. The basic frame work of these molecules is not only present in a variety of natural products, but it also serves as a starting material for more complex structures like thienopyridines, thienopyrimidines, pyrido thienopyrimidines, imidazo thienopyrimidines, which have shown extensive inhibitory properties in a variety of biological targets. In addition to industrial applicability of thiophenes, great interest has built up in the field of automated combinatorial synthesis. Gewald's synthetic method for preparation of thiophenes is highly efficient and comparable with modern methods.

The six possible thienopyridine systems (thieno (x, y-z) pyridines) fall into two groups, those that are analogs of quinolines, and of isoquinolines. Interest on thienopyridines has increased recently due to a theoretical interest in the behavior of systems that contain, fused together, a π-excessive and a π-deficient ring. The search for pharmacologically active substances has led to the synthesis of analogs of various quinolines and isoquinolines where the benzene ring is replaced by a thiophene nucleus. Unlike their benzene counterparts, thienopyridines do not occur widely in nature. The [c]-fused systems are stronger bases than those that are [h]-fused.

The present invention deals with synthesis and biological cytotoxic screening of new fused thienopyridine derivatives for antiinflammatory, antiarthritic and cytotoxic and antidiabetic activities. While a few thienoquinolines are reported as acetylcholinesterase inhibitors, to the best of our knowledge, there has been no medicinal chemistry effort to develop thienopyridine based antiinflammatory, antiarthritic and anticancer and antidiabetic agents.

### OBJECTS OF THE INVENTION

The primary object of the invention is to provide novel thienopyridine compounds that possess new and/or improved therapeutic activities.

It is a further object of the invention to provide novel thienopyridine compounds that possess, inter alia, anti-inflammatory, anti-diabetic, anti-cancer, anti-arthritic and adenoside antagonistic activity.

It is another object of the invention to provide thienopyridine based agents for the treatment of inflammation, diabetes, cancer, arthritis, and asthma.

It is another object of the invention to provide methods for the synthesis of thienopyridine compounds or salts thereof which are useful in the treatment of inflammation, diabetes, cancer, arthritis, and asthma.

Yet another object of the invention is to provide methods for the inhibition of TNF-α activity using thienopyridines or salts thereof, or prodrugs thereof.

Yet another object of the invention is to provide novel thienopyridines and/or salts thereof, and/or prodrugs thereof, which are useful in the diagnosis, prevention and/or treatment of inflammation, arthritis, cancer, asthma and diabetes.

### STATEMENT OF INVENTION

The above and other objects of the invention are achieved by providing novel thienopyridine compounds of the formula I below

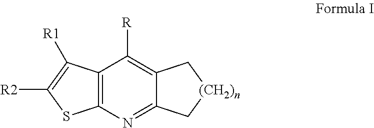

where n is 1, 2, 3 or 4; R1 and R2 are independently selected from the group consisting of CH₃, alkyl and aryl; or R1+R2 is selected from cyclopentyl, cyclohexyl, cycloheptyl, bicycloalkyl, and alkyl of more than 2 carbon chain; R is selected from the group consisting of amine, substituted amine, amino acids, sulfonamide, sulfonyl alkyl, alkyl or cycloalkyl, aryl, hydroxamate, and amino heterocyclic moieties; wherein the heterocyclic moiety is selected from imidazole, triazole, tetrazole, pyridine, benzimidazole, quinazoline, quinoline, thiophene, thienopyrimidine, thienopyridine, indole, pyrrole acridine, and benzofuran and pharmaceutically acceptable salts or derivatives thereof.
If desired, the heterocyclic moieties may bear substituents selected from —H, —(C₁-C₃) alkyl, —O(C₁-C₃) alkyl, F, —CF₃, —NH₂, —N(CH₃), —N(CH₃)₂, —SH, —SCH₃, —SCH₂CH₃ and any combination thereof.

In a further embodiment, the present invention also provides isomers of compounds of the general formula I, inclusive of their stereoisomeric forms, polymorphs, acid addition salts, base addition salts, and prodrugs thereof.

In yet another embodiment, the acid addition salts are selected from the group consisting of acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, napthelene-2-benzoate, isobutyrate, phenylbutyrate, b-hydroxybutyrate, butyne-1-4-dioate, hexyne-1-4-dioate, caprate, caprylate, cinnamate, citrate, formate, fumerate, glycollate, heptanoate, hippurate, lactate, maleate, malate, hydroxymaleate, malonate, madelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, ethanesulfonate, naphthalene-1-sulfonate, naphthalene-2-sulfonate, p-toluenesulfonate, xylenesulfonate, tartarate, and the like. Preferred salts are the hydrochloride, hydrobromide, citrate and oxalate.

In a further embodiment of the invention, the basic addition salts are formed from inorganic bases selected from the group consisting of sodium, potassium, lithium, calcium, aluminium, ammonium, barium, zinc, magnesium and the like, or organic bases selected from the group consisting of N—N′-dibenzylethelynediamine, choline, diethanolamine, ethelenediamine, N-methylglucamine, triethylamine, dimethylamine, procaine salts and the like, and salts of amino acids such as arginate.

In a further embodiment of the invention, the prodrugs are selected from those formed by conjugation of compounds of formula I with sugar moieties with suitable spacers, or alkyl esters obtained by reaction of the parent acid with a suitable alcohol, or amides obtained by reaction of parent acidic compound with a suitable amine.

In another embodiment of the invention, aryl includes phenyl, biphenyl, benzyl, naphthyl, anthryl, phenanthryl, fluorenyl and indenyl.

In yet another embodiment, the heterocycle is selected from the group consisting of imidazole, triazole, tetrazole, indole, pyrrole and the like.

In another embodiment of the invention, the heterocycle has one or more heteroatoms selected from O, S and N in the aromatic ring.

In a further embodiment of the invention, the compounds of general formula I are selected from the group consisting of 2,3-dimethyl-6,7-dihydro-5H-cyclopenta[b]thieno[3,2-e]pyridin-4-amine 2,3-dimethyl-5,6,7,8-tetrahydrothieno[2,3-b]quinolin-4-amine 2,3,7-trimethyl-5,6,7,8-tetrahydrothieno[2,3-b]quinolin-4-amine 2,3-dimethyl-6,7,8,9-tetrahydro-5H-cyclohepta[b]thieno[3,2-e]pyridin-4-amine 2,3,6,7,8,9-hexahydro-1H-benzo[4,5]thieno[2,3-b]cyclopenta[e]pyridin-10-amine 1,2,3,4,7,8,9,10-octahydrobenzo[4,5]thieno[2,3-b]quinolin-11-amine 8-methyl-1,2,3,4,7,8,9,10-octahydrobenzo[4,5]thieno[2,3-b]quinolin-11-amine 2,3,4,7,8,9,10,11-octahydro-1H-benzo[4,5]thieno[2,3-b]cyclohepta [e]pyridin-12-amine 1,2,3,6,7,8-hexahydrocyclopenta[b]cyclopenta[4,5]thieno[3,2-e]pyridin-9-amine 2,3,6,7,8,9-hexahydro-1H-cyclopenta[4,5]thieno[2,3-b]quinolin-10-amine 7-methyl-2,3,6,7,8,9-hexahydro-1H-cyclopenta[4,5]thieno[2,3-b]quinolin-10-amine 1,2,3,6,7,8,9,10-octahydrocyclohepta[b]cyclopenta[4,5]thieno[3,2-e]pyridine-11-amine 2,3,6,7,8,9,10,11-octahydro-1H-cycloocta[b]cyclopenta[4,5]thieno[3,2-e]pyridin-12-amine 1,2,3,6,7,8,9,10-octahydrocyclohepta[4,5]thieno[2,3-b]cyclopenta[e]pyridin-11-amine 2,3,4,7,8,9,10,11-octahydro-1H-cyclohepta[4,5]thieno[2,3-b]quinolin-12-amine.

The present invention also provides a method for the synthesis of the compounds of formula I which comprises first synthesizing the precursor 2-amino 3-cyano thiophenes and then reacting with cyclic ketone under conditions suitable to obtain the corresponding product of formula I.

In one embodiment of the invention, the method comprises carrying out the reaction in the presence of zinc chloride to form a complex, followed by treatment with a base to release the product compound from the zinc chloride complex, followed by separating and purification of the precipitate.

In another embodiment of the invention, the thiophene and the cyclic ketone are reacted at a molar ratio of 1:2.

In another embodiment of the invention, the base used in the above process is NaOH.

In another embodiment of the invention, the 2-amino, 3-cyano thiophene is prepared by reacting sulphur, melanonitrile and respective ketone in the presence of an alcohol under stirring.

In yet another embodiment of the invention, the conversion of 2-amino, 3-cyano thiophene to the title compound is carried out by heating under reflux.

In yet another embodiment of the invention the compound of formula I is reacted with an equimolar amount or an excess of acid in a neat or in a suitable inert solvent to form the corresponding acid addition salt.

In yet another embodiment of the present invention wherein the said acid is selected from the group consisting of hydrochloric, hydrobromic, hydroiodic, nitric, sulfuric, phosphoric, hypophosphoric, aliphatic mono and dicarboxylic acid, phenyl substituted alkanoic acids, hydroxyalkanoic and hydroxyalkandioic acids, aromatic acids, aliphatic and aromatic sulfonic acids.

In yet another embodiment of the present invention wherein the compound of formula I is reacted in an inert suitable solvent or a neat solvent with an equimolar or excess amount of a base to form the corresponding base addition salt.

In yet another embodiment of the present invention wherein the said base is selected from sodium, potassium, lithium, calcium, aluminium, ammonium, barium, zinc, magnesium, N—N′-dibenzylethelynediamine, choline, diethanolamine, ethelenediamine, N-methylglucamine, triethylamine, dimethylamine, and procaine, and amino acids to obtain the respective basic addition salts.

In another embodiment of the present invention wherein the acidic addition salt of compound of formula I salt is formed by sending the dry acidic gas into the methanolic solution of the compound.

In another embodiment of the present invention wherein the prodrug of a compound of formula (I) is obtained by conjugation of compounds of formula (I) with sugar moieties adding suitable spacers, or alkyl esters prepared by the reaction of the parent acidic compound with a suitable alcohol, or amides prepared by reaction of the parent acid compound with suitable amine.

In an embodiment of the present invention a pharmaceutical composition comprising the compound of formula I

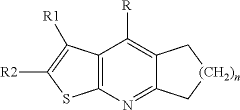

where n is 1, 2, 3 or 4,
R1 and R2 are independently selected from the group consisting of CH₃, alkyl and aryl; or
R1+R2 is selected from cyclopentyl, cyclohexyl, cycloheptyl, bicycloalkyl, and alkyl of more than 2 carbon chain;
R is selected from the group consisting of amine, substituted amine, amino acids, sulfonamide, sulfonyl alkyl, alkyl or cycloalkyl, aryl, hydroxamate, and amino heterocyclic moieties;
wherein the said heterocyclic moiety is selected from imidazole, triazole, tetrazole, pyridine, benzimidazole, quinazoline, quinoline, thiophene, thienopyrimidine, thienopyridine, acridine, indole pyrrole and benzofuran and pharmaceutically acceptable salts or derivatives thereof
with a conventional active agent for treatment of diabetes, cancer, arthritis or inflammation.

In yet another embodiment of present invention a pharmaceutical composition comprising the compound of formula I or a salt or ester or prodrug form thereof with one or more pharmaceutically acceptable excipients.

In yet another embodiment of present invention a pharmaceutical composition comprising the compound of formula I with a conventional active agent for treatment of diabetes, cancer, arthritis or inflammation.

In yet another embodiment of present invention wherein the pharmaceutical composition comprises an additional active wherein the additional active selected from the group consisting of alkylating agents, antimetabolites, antibiotics, immunomodulating agents, nucleotide derivatives, cyclin dependent kinase inhibitors, interferon like agents and histone deacytalase inhibitors.

In yet another embodiment of present invention wherein the pharmaceutical composition comprises an additional active wherein the additional active is selected from the group consisting of COX-II inhibitors such as nimuselide, celocoxib, etorocoxib, and valdicoxib.

In yet another embodiment of present invention wherein the pharmaceutical composition comprises an additional active wherein the additional active is selected from the group consisting of sulphonylureas, Biguanides, Meglitinides, Glitazones, and α-Glucosidase Inhibitors.

In yet another embodiment of present invention wherein the pharmaceutical composition comprises a pharmaceutically acceptable excipients wherein the pharmaceutically acceptable excipients are selected from the group consisting of pharmaceutically acceptable carrier or diluent.

In yet another embodiment of present invention wherein the pharmaceutical composition comprises a carrier or diluents wherein the carrier or diluents is selected from the group consisting of water, salt solutions, alcohols, polyethylene glycols, polyhydroxy ethoxylated castor oil, peanut oil, olive oil, gelatine, lactose, sucrose, cyclodextrin, amylose, magnesium stereate, talc, agar, silicic acid, fatty acids, fatty acid amines, fatty acid monoglycerides, fatty acid diglycerides, polyoxyethylene, hydroxymethylcellulose, and polyvinylpyrrolidine.

The invention also provides a method for the treatment of cancer, diabetes, arthritis and inflammation or to prevent diseases associated with it in a subject by administering therapeutically effective amount of a compound of the formula (I):

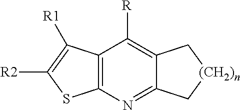

wherein n=1, 2, 3, 4
R1=R2=CH₃, alkyl, aryl or R1+R2=—(CH₂)₃—, —(CH₂)₄—, —(CH₂)₅—R=Amine, substituted amine, amino acids, sulfonamide, sulfonyl alkyl, alkyl or cycloalkyl, aryl, hydroxamate, amino heterocyclic moieties. The heterocyclic moiety may be selected from but it is confined to imidazole, triazole, tetrazole and pyridine, benzimidazole, quinazoline, quinoline, thiophene, thienopyrimidine, thienopyridine, acridine, indole, pyrrole, benzofuran. These heterocyclic moieties may bear substituents selected from —H, —(C₁-C₃) alkyl, —O(C₁-C₃) alkyl, F, —CF₃, —NH₂, —N(CH₃), —N(CH₃)₂, —SH, —SCH₃, —SCH₂CH₃ and combination there of.
or a pharmaceutically acceptable salt or derivative thereof.

The present invention also provides a method for use of these compounds in treatment of cancer, inflammation, diabetes and arthritis. For example, the method includes treatment of skin, colon, lung cancer, particularly, but not limited to hormone dependent cancer. The method includes administering a therapeutically effective amount of compounds of present invention, or a derivative or pharmaceutical salt there of, to subject in need of treatment. These compounds can be used along with other standard drugs in combination therapy for better efficacy. Also provided is a method for the prevention of cancer, inflammation, diabetes and arthritis in subjects who are susceptible to developing cancer, inflammation, diabetes and arthritis comprising administering a therapeutically effective amount of compounds of present invention, or a derivative or pharmaceutical salt thereof.

In other embodiments, the invention includes compositions containing a pharmaceutically acceptable amount of compound of formula I or any derivative thereof, inclusive of stereoisomers, salts, polymorphs, esters, and prodrugs, along with a pharmaceutically acceptable carrier.

In another embodiment, the invention includes the use of compound of formula I when used in combinational therapy along with other known actives for the treatment of cancer, diabetes, arthritis, inflammation.

### BRIEF DESCRIPTION OF THE ACCOMPANYING DRAWINGS

FIG. 1: Carrageenan induced inflammation model. The symbol # represents no statistical significance at p>0.05 when compared with indomethacin at dose of 10 mg/kg. The symbol a indicates significant difference (p<0.01) between compounds and standard indomethacin at the dose of 10 mg/kg. Values are expressed as Mean±S.E.M, (n=3).

FIG. 2: Carrageenan induced inflammation model. The symbol # represents no statistical significance at p>0.05 when compared with indomethacin at dose of 10 mg/kg. The symbol a indicates significant difference (p<0.01) when compared with standard indomethacin at the dose of 10 mg/kg. Values are expressed as Mean±S.E.M, (n=3).

FIG. 3: Carrageenan Induced paw edema—Dose Response study. The symbol # represents no statistical significance at p>0.05 when compared with indomethacin at dose of 10 mg/kg. The symbol a indicates significant difference (p<0.01) when compared with standard indomethacin at dose of 10 mg/kg. Values are expressed as Mean±S.E.M, (n=6).

FIG. 4: Dextran induced paw edema—Dose response study. The symbol # represents the no statistical significance difference (p>0.05) when compared with indomethacin at dose of 10 mg/kg. The symbol a indicates significant difference (p<0.01) when compared with standard indomethacin at dose of 10 mg/kg. Values are expressed as Mean±S.E.M, (n=6).

FIG. 5: Arachidonic acid Induced Paw Edema Model—Dose response study. The symbol # represents the no statistical significant difference (p>0.05) when compared with indomethacin at dose of 10 mg/kg. The symbol a and b represents significant difference (p<0.01 and P<0.05) when compared with standard drug indomethacin at dose of 10 mg/kg. Values are expressed as Mean±S.E.M, (n=6).

FIG. 6: Cotton pellet granuloma—wet weight. Graph shows granuloma tissue weight for the compounds BN-4, BN-14, BN-16 at dose of 100 mg/kg. The symbol # represents no statistical significance difference at p>0.05 when compared with indomethacin at dose of 10 mg/kg. Values are expressed as Mean±S.E.M, (n=6).

FIG. 7: Cotton pellet granuloma—Dry weight. Graph shows granuloma tissue weight for the compounds BN-4, BN-14, BN-16 at the dose of 100 mg/kg. The symbol # represents the no statistical significance difference at p>0.05 when compared with indomethacin at the dose of 10 mg/kg. Values are expressed as Mean±S.E.M, (n=6).

FIG. 8: TNF-a assay. Inhibitory effect of BN-4, BN-14, BN-16 on production of TNF-a from murine macrophage cell line is seen. Values are expressed as Mean±S.E.M. * represents significant statistical difference (p<0.01) compared with LPS control.

FIG. 9: IL-1β assay. The inhibitory effect of BN-4, BN-14, BN-16 on the production of IL-1β from murine macrophage cell line is seen. Values are expressed as Mean±S.E.M. The symbols * represents significant statistical difference comparing with LPS control and # indicates no statistical difference compared with LPS control.

FIG. 10: Nitrite oxide estimation in macrophage cell line. The inhibitory effect of BN-4, BN-14, BN-16 on the production of Nitric oxide from Murine macrophage cell line is seen. The symbol * represents significant statistical difference compared with LPS control. Values are expressed as Mean±S.E.M.

FIG. 11: Measurement of Nitrite —SNP method. The inhibitory effect of BN-4, BN-14, BN-16 on the production of Nitric oxide in SNP method is seen. The symbol * represents significant statistical difference compared with control.

FIG. 12: Body Weight increase in FCA induced arthritis study for the test compounds on termination day. * Indicates significant difference P<0.01 comparing with Normal control. # indicated no statistical difference when compared with Normal control (p>0.05).

FIG. 13: Arthritic index of Thienopyridine derivative at different time intervals on FCA induced arthritis study.

FIG. 14: Effect of Thienopyridine derivatives on Freunds complete adjuvant induced arthritis model in rats. Graph shows paw volumes for the compounds BN-4, BN-14, BN-16 at the dose of 100 mg/kg. Values are expressed as Mean±S.E.M, (n=6). * Represents P>0.01 when compared with arthritic control group of rats. a represents significant statistical difference when compared with arthritic control group at dose of 2.5 mg/kg. # represents significant statistical difference p<0.05) comparing with arthritic control.

FIG. 15: Effect of Thienopyridine derivatives on Freunds complete adjuvant induced arthritis model in rats. Graph shows WBC index on 21-st day for the compounds BN-4, BN-14, BN-16 at the dose of 100 mg/kg. Values are expressed as Mean±S.E.M, (n=6).** Indicates no statistical difference when compared with arthritic control.* represents significant difference when compared with arthritic control group.

FIG. 16: Standard graph for the estimation of IL-1 Beta in paw tissue of rats.

FIG. 17: Standard graph for the estimation of TNF-Alfa in paw tissue, of rats.

FIG. 18: Estimation of IL-1 Beta in paw tissue —FCA study. Effect of Thienopyridine derivatives BN-4, BN-14, BN-16 on IL1-β expression in the paw tissue of arthritic rats on 21-st day in FCA induced arthritis study. Results are shown as the Mean±S.E.M. for six animals in each group. * P<0.05 when compared with arthritic control rats and normal control rats.

FIG. 19: Estimation of TNF Alfa in paw tissue —FCA study. Effect of treatment with compounds BN-4, BN-14, BN-16 on the concentration of TNF-∝ in the hind paw tissue of arthritic rats in FCA induced arthritis study. Results are shown as the Mean±S.E.M. for six animals in each group. * P<0.01 when compared with arthritic control rats.

FIG. 20: Standard graph for IL1-β in serum of rats for both FCA and Collagen induced arthritis models.

FIG. 21: Effect of Thienopyridine derivatives BN-4, BN-14, BN-16 on the expression of IL1-β in the serum of arthritic rats in FCA induced arthritis study. Results are shown as the Mean±S.E.M. for six animals in each group. * P<0.01 when compared with arthritic control.

FIG. 22: Effect of different Thioenopyridine derivatives on Yeast α-Glucosidase inhibition assay. * indicates no significance difference (p>0.05) when compared with acarbose (1.25 mg/ml).^{a} indicates significance difference (p<0.01) compared with standard (Acarbose 5 mg/ml).

FIG. 23: Effect of different Thioenopyridine derivatives on Yeast α-Glucosidase inhibition assay. * indicates no significance difference (p>0.05) when compared with standard, and the compound and BN-14 is comparable with standard Acarbose at dose of 5 mg/ml .^{a} indicates significance difference (p<0.01) compared with standard (Acarbose).

FIG. 24: Effect of different Thinopyridine derivatives on Mammalian α-Glucosidase inhibition assay. a indicates significance difference (p<0.01) compared with standard (Acarbose 5 and 2.5 mg/ml)

FIG. 25: Effect of different Thioenopyridine derivatives on Mammalian α-Glucosidase inhibition assay. # indicates p>0.05 no significance difference compared with standard (Acarbose 2.5 mg/ml)).a indicates p<0.01 significance difference when compared with acarbose (2.5 and 5 mg/ml).b indicates p<0.05 significant difference compared with acarbose (2.5 mg/ml)

FIG. 26: Standard graph for the maltose standard related to α-amylase inhibition.

FIG. 27: Effect of Different synthetic compounds on α Amylase inhibition assay. Graph shows amount of maltose release after end of the reaction in the Assay. # indicates p>0.05 no significance when compared with control maltose release. * indicates significance difference compared with control maltose release. a indicates p<0.01 significance difference compared with control but maltose release was more than control in the test compound (BN-18).

FIG. 28: Effect of Different synthetic compounds on a Amylase inhibition assay. Graph shows amount of maltose release after end of the reaction in the Assay. # indicates p>0.05 no significance when compared with control maltose release. * indicates significance difference compared with control maltose release.

FIG. 29: Effect of synthetic compounds BN-13, BN-14, BN-15, and BN-17 on Starch loaded model in Wistar rats. Graph shows percentage increase in glucose levels at different time points. * Indicates p<0.01 statistical significant difference when compared with diabetic control. Values are expressed as Mean±S.E, n=6.

### DETAILED DESCRIPTION OF INVENTION

The present invention provides new possible small molecule TNF-α inhibitors, α-glucosidase and α-amylase inhibitors in the form of fused thienopyridine derivatives and methods of using those compounds. The present invention encompasses thienopyridines, as well as salts, esters and derivatives and related compounds. The fused thienopyridines have the following structure

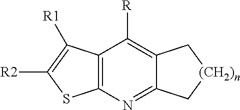

wherein n=1, 2, 3, 4
R1=R2=CH₃ or R1+R2=Alkyl of more than 2 carbon chain, cyclopentyl, cyclohexyl, cycloheptyl, bicycloalkyl, Aryl,
R=Amine, substituted amine, amino acids, sulfonamide, sulfonyl alkyl, alkyl or cycloalkyl, aryl, hydroxamate, amino heterocyclic moieties. The heterocyclic moiety may be selected from but it is confined to imidazole, triazole, tetrazole and pyridine, benzimidazole, quinazoline, quinoline, thiophene, thienopyrimidine, thienopyridine, acridine, benzofuran, indole and pyrrole. These heterocyclic moieties may bear substituents selected from —H, —(C₁-C₃) alkyl, —O(C₁-C₃) alkyl, F, —CF₃, —NH₂, —N(CH₃), —N(CH₃)₂, —SH, —SCH₃, —SCH₂CH₃ and combination thereof, or a pharmaceutically acceptable salt or derivative thereof.

The term aryl, used alone or in combination with other terms such as alkylaryl, haloaryl or haloalkylaryl includes such aromatic rings as phenyl, biphenyl and benzyl, as well as fused aryl radicals such as napthyl, anthryl, phenanthryl, fluorenyl and indenyl on so forth. The term “heterocycle” encompasses the aryls that have one or more heteroatoms. Such as O, N or S in the aromatic ring. Examples of heterocycles include imidazole, triazole, tetrazole, indole, pyrrole and so on.

As used herein the term “stereoisomer” refers to a compound made up of the same atoms bonded by the same bonds but having different three-dimensional structures, which are not interchangeable. The stable three-dimensional structures are called configurations. As used herein, the term “enantiomer” refers to two stereoisomers whose molecules are non superimposable mirror images of one another. The term “chiral center” refers to a carbon atom to which four different groups are attached. The term “diastereoisomer” refers to stereoisomers that are not enantiomers. Two diastereoisomers, which have different configuration at only one chiral center, are referred to herein as “epimers”. The term “racemate”, “racemic mixture” or “racemic modification” refers to a mixture of equal parts of enantiomers. In addition, “geometrical isomer” related to compounds containing double bond and the four atoms directly attach to them are all in the plane and that rotation around the double bond is prevented. The two groups attached to each carbon atom are ranked by Cahn-Ingold-Prelog system; the “Z isomer” refers to that isomer with the two higher ranking groups on the same side of the double bond. The “E isomer” refers to that isomer with the two higher ranking groups on the opposite side of the double bond.

Additionally, those skilled in the art will recognise that stereocenters exist in compounds of formula (I). Accordingly the present invention includes all possible stereoisomers including optical and geometric isomers of formula (I). It further includes not only racemic compounds, or racemic mixtures thereof, but also optically active isomers. When a compound of formula (I) is desired as a single enantiomer, it may be either by resolution of final product or by a stereospecific synthesis from either optically pure starting material or any convenient intermediate, and is additionally, intended to include all tautomeric forms of the compounds. These terms and methods for identifying and selecting desired compounds are well known in the art for example, diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from the appropriate stereochemically pure starting materials, or by using stereoselective reactions.

The compounds of present invention form pharmaceutically acceptable acid or base addition salts with a wide variety of organic and inorganic acids and bases, and include the physiologically acceptable salts which are often used in pharmaceutical chemistry such salts are also part of this invention. Typical inorganic acids used to form such salts include hydrochloric, hydrobromic, hydroiodic, nitric, sulfuric, phosphoric, hypophosphoric, and the like. Salts derived from organic acids aliphatic mono and dicarboxylic acid, phenyl substituted alkanoic acids, hydroxyalkanoic and hydroxyalkandioic acids, aromatic acids, aliphatic and aromatic sulfonic acids may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, napthelene-2-benzoate, isobutyrate, phenylbutyrate, b-hydroxybutyrate, butyne-1-4-dioate, hexyne-1-4-dioate, caprate, caprylate, cinnamate, citrate, formate, fumerate, glycollate, heptanoate, hippurate, lactate, maleate, malate, hydroxymaleate, malonate, madelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, ethanesulfonate, naphthalene-1-sulfonate, naphthalene-2-sulfonate, p-toluenesulfonate, xylenesulfonate, tartarate, and the like. Preferred salts are the hydrochloride, hydrobromide, citrate and oxalate.

Typical bases used to form pharmaceutically acceptable addition salts would be inorganic bases such as, sodium, potassium, lithium, calcium, aluminium, ammonium, barium, zinc, magnesium and the like. Additionally, organic bases may be utilized to form salts fro example N—N′-dibenzylethelynediamine, choline, diethanolamine, ethelenediamine, N-methylglucamine, triethylamine, dimethylamine, procaine salts and the like. Also included are salts of aminoacids such as arginate and the like.

Pharmaceutically acceptable acid or base salts are typically formed by reacting a compound of formula (I) with an equimolar or excess amount of acid or base in a neat or in a suitable inert solvent. The formed salts are further processed and purified by known methods. The salts can also be formed by sending the dry acidic gas into the methanolic solution of the compound.

The compounds of the present invention might be in a prodrug form. A prodrug is generally a pharmacologically inactive derivative of a parent drug molecule that requires spontaneous or enzymatic transformation within the body in order to release an active drug, and that has improved pharmacokinetic properties over parent drug molecule. Therefore, the prodrug of compound of general formula (I) has chemically or metabolically cleavable groups and which readily undergoes chemical changes under physiological conditions to provide a compound of formula (I) in vivo. Prodrugs include conjugates of compounds of formula (I) with sugar moieties adding suitable spacers, alkyl esters prepared by reaction of parent acidic compound with a suitable alcohol, or amides prepared by reaction of parent acid compound with suitable amine.

By “treating” is meant curing, ameliorating or tampering the severity of the cancer or the symptoms or the effects associated there with. The terms “treating”, “treatment” and “therapy” as used herein refer to curative, prophylactic, and preventive therapy.

“Preventing” or “prevention” means preventing the occurrence of the diseases mentioned above or tampering the severity of the diseases with subsequent to the administration of the compositions. This prevents the onset of a clinically evident unwanted indications altogether or preventing the onset of a preclinically evident stage of unwanted autoimmune diseases in individuals at risk. Also intended to be encompassed by this definition is the prevention of metastasis of malignant cells or to arrest or reverse the progression of malignant cells. This includes prophylactic treatment of those at risk of developing precancers and cancers.

The terms “therapeutically effective” and “pharmacologically effective” are intended to qualify the amount of each agent, which will achieve the goal of improvement in disease severity and frequency of incidence over treatment of each agent by itself. While avoiding adverse side effects associated with alternative therapies.

The term “subject” for purposes of treatment includes any human or animal subject having a disease involving TNF-α, alpha glucosidase and alpha-amylase. For methods of prevention the subject is any human or animal subject, and preferably is a human subject at risk of developing a disease mentioned there of. The subject may be at risk due to exposure to carcinogenic agents, inflammatory conditions being genetically predisposed to disorders characterized by unwanted, rapid cell proliferation and so on. Besides being useful for human treatment, compounds of the present invention are useful for veterinary treatment of mammals, including companion animals and farm animals, such as, but not limited to dogs, cats, horses, cows, sheep, and pigs. Preferably, subject means a human.

Routes of administration for the compounds of formula (I) are oral, subcutaneous, intramuscular, topical or intravenous or by any route which delivers the therapeutically effective amount of active agent to the organ or tissue or site to be treated. It will be appreciated that different dosages may be required for treating different diseases mentioned there of. Additionally the compounds of present invention may be delivered to the target site or tumor novel techniques known to the art like antibody conjugation, pH sensitive polymer implants and so on. The preferred route of administration is oral.

By “pharmaceutically effective dose” is meant an amount of a pharmaceutical compound or composition having a therapeutically relevant effect in the frame of treatment and/or prevention of disease conditions. The dosage also depends upon variety of factors, including age, weight, sex, medical condition of the patient, the severity of disease, the route and frequency of administration, potency of the compound employed, the site of the proliferation, as well as pharmacokinetic properties of the compound being administered, adverse and toxicological effects of the compound being used and interaction of the present invention compound with other agents. Generally the dosage of compounds administered locally rather than systemically and for prevention rather than treatment will be lower. Such treatments may be administered as often as necessary and for the period of time judged necessary by the treating physician and veterinarian. One of the skill in the art will appreciate that the dosage regime or therapeutically effective amount of the compound to be administered may need to optimized for each subject. A typical daily dose for the compounds will contain nontoxic dosage level from about 1 mg to about 800 mg/day of a compound. Preferred daily doses generally will be from about 1 mg to 200 mg/day. Most preferred doses range may constitute 1 mg, 2 mg, 5 mg, 10 mg, 20 mg, 30 mg, 40 mg, 50 mg, 60 mg, 70 mg, 90 mg and 100 mg. The compounds or compositions (including combination with additional agent) of the present invention may be administered in a single dose, or the total daily dosage may be administered in individual doses divided into two, three, or four times daily. Similarly, the treatment can be adapted to administer the compounds or compositions (including combinations) of the invention in a single weekly or monthly dose.

The term “used in combination with” mean that administration of compounds of formula (I) simultaneously or consecutively with one or more other pharmaceutical agent, simultaneously refers to a co-administration. In this case, the separate components of the combination can be mixed to form a single composition prior to being administered, or can be administered at the same time to the patient. It is also possible to administer them consecutively that is to say one after the other, irrespective of which component of the combination according to the invention is administered first. It is possible to use a mode of administration, which is staggered over time or is intermittent and which stops and restarts at intervals which may or may not be regular. It is pointed out that the routes and sites of administration of the two components can be different. The time interval between the administration is not critical and can be defined by the skilled person.

Examples for additional agents of combination therapy are other large numbers of antineoplastic agents available in market, in clinical trials, or in preclinical evaluation, which could be selected for treatment of cancers or other neoplasias by combination drug chemotherapy. These agents fall into several major categories, namely alkylating agents, antimetabolites, antibiotics, immunomodulating agents, nucleotide derivatives, cyclin dependent kinase inhibitors, interferon like agents and histone deacytalase inhibitors.

Other preferred agents for combination therapy are COX-II inhibitors, examples for such category of compounds include nimuselide, celocoxib, etorocoxib, valdicoxib and like. Other preferred agents for combination therapy are sulphonylureas, Biguanides, Meglitinides, Glitazones, and α-Glucosidase Inhibitors

In another embodiment, the invention further provides a composition comprising at least one compound of the invention and pharmaceutically acceptable carrier or diluent. These pharmaceutical compositions may be prepared by conventional techniques. Typical compositions of the present invention are associated with a pharmaceutically acceptable excipients which may be a carrier or a diluent or be diluted by a carrier, or enclosed with in a carrier which can be in the form of a capsule, sachet, tablets, aerosols, solutions, suspensions, injectables or other compositions. In making the combination products, conventional techniques for the preparation of pharmaceutical composition may be used. For example, the active compound will usually be mixed with a carrier or a diluent, or diluted by carrier or a diluent, or enclosed within a carrier or diluent which may be in the form of a injectable, capsule, sachet, tablets, aerosols, solutions, suspensions or other compositions. When the other carrier serves as a diluent, it may be solid, semisolid, or liquid material, which acts as a vehicle, excipient, or medium for the active compound.

Some examples of suitable carriers or diluents are without being limited, water, salt solutions, alcohols, polyethylene glycols, polyhydroxy ethoxylated castor oil, peanut oil, olive oil, gelatine, lactose, sucrose, cyclodextrin, amylose, magnesium stereate, talc, agar, silicic acid, fatty acids, fatty acid amines, fatty acid monoglycerides, fatty acid diglycerides, polyoxyethylene, hydroxymethylcellulose, and polyvinylpyrrolidine.

Tablets and capsules represent the most advantageous oral dosage unit form, in which case solid pharmaceutical carriers are obviously employed. The compounds also can be formulated are elixirs or solutions for convenient oral administration. The active compounds also formulated as appropriate solutions for parenteral administration for example by intravenous, intramuscular, and subcutaneous. Additionally the carrier or diluent may include any sustain release material known in the art, such as glyceryl monostereate or glyceryl distereate. In one embodiment the active compounds are incorporated into controlled release formulation, including implants and microencapsulated delivery systems. The compounds of the present invention can also be administered in the liposomes or nanoparticle delivery systems. The composition can be so constituted that they release the active ingredient only or preferably in a particular physiological location, possibly over a period of time.

More specifically, the invention concerns compounds falling within the scope of the formula (I), including but not limited to the following or their pharmaceutically acceptable salts:
2,3-dimethyl-6,7-dihydro-5H-cyclopenta[b]thieno[3,2-e]pyridin-4-amine (BN-1)
2,3-dimethyl-5 ,6 ,7 ,8-tetrahydrothieno[2 , 3-b]quinolin-4-amine (BN-2)
2,3,7-trimethyl-5 ,6 ,7 ,8-tetrahydrothieno[2,3-b]quinolin-4-amine (BN-3)
2,3-dimethyl-6,7,8,9-tetrahydro-5H-cyclohepta[b]thieno[3,2-e]pyridin-4-amine (BN-4)
2,3,6,7,8,9-hexahydro-1H-benzo[4 , 5]thieno[2,3-b]cyclopenta[e]pyridin-10-amine (BN-5)
1,2,3,4,7,8,9,10-octahydrobenzo[4,5]thieno[2 , 3-b]quinolin-11-amine (BN-6)
8-methyl-1,2,3,4,7,8,9,10-octahydrobenzo[4,5]thieno[2 , 3-b]quinolin-11-amine (BN-7)
2,3,4,7,8,9,10,11-octahydro-1H-benzo[4,5]thieno[2,3-b]cyclohepta [e]pyridin-12-amine (BN-8)
1,2,3,6,7,8-hexahydrocyclopenta[b]cyclopenta[4,5]thieno[3,2-e]pyridin-9-amine (BN-13)
2,3,6,7,8,9-hexahydro-1H-cyclopenta[4,5]thieno[2,3-b]quinolin-10-amine (BN-14)
7-methyl-2,3,6,7,8,9-hexahydro-1H-cyclopenta[4,5]thieno[2,3-13]quinolin-10-amine (BN-15)
1,2,3,6,7,8,9,10-octahydrocyclohepta[b]cyclopenta[4,5]thieno[3,2-e]pyridine-11-amine (BN-16)
2,3,6,7,8,9,10,11-octahydro-1H-cycloocta[b]cyclopenta[4,5]thieno[3,2-e]pyridin-12-amine (BN-17)
1,2,3,6,7,8,9,10-octahydrocyclohepta[4,5]thieno[2,3-b]cyclopenta[e]pyridin-11-amine (BN-18)
2,3,4,7,8,9,10,11-octahydro-1H-cyclohepta[4,5]thieno[2,3-b]quinolin-12-amine (BN-19).

We designed a library of thienopyridines as shown in formula (I). The synthesis of target compounds was carried as procedure outlined in scheme 1

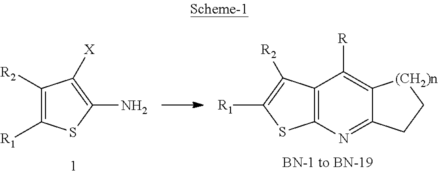

The following compounds were synthesized from scheme I

| Compd code | R1 | R2 | n | R |
|---|---|---|---|---|
| BN-1 | CH₃ | CH₃ | 1 | NH₂ |
| BN-2 | CH₃ | CH₃ | 2 | NH₂ |
| BN-3 | CH₃ | CH₃ | 2 (m-CH₃) | NH₂ |
| BN-4 | CH₃ | CH₃ | 3 | NH₂ |

| | | | |
|---|---|---|---|
| BN-5 | —(CH₂)₄— | 1 | NH₂ |
| BN-6 | —(CH₂)₄— | 2 | NH₂ |
| BN-7 | —(CH₂)₄— | 2 (m-CH₃) | NH₂ |
| BN-8 | —(CH₂)₄— | 3 | NH₂ |
| BN-13 | —(CH₂)₃— | 1 | NH₂ |
| BN-14 | —(CH₂)₃— | 2 | NH₂ |
| BN-15 | —(CH₂)₃— | 2 (m-CH₃) | NH₂ |
| BN-16 | —(CH₂)₃— | 3 | NH₂ |
| BN-17 | —(CH₂)₃— | 4 | NH₂ |
| BN-18 | —(CH₂)₅— | 1 | NH₂ |
| BN-19 | —(CH₂)₅— | 2 | NH₂ |

The desired compounds from scheme 1 were prepared by the following protocol.

The compounds in the first step or otherwise starting materials were prepared using Gewald procedure. Sulphur (0.01 mole), melanonitrile (0.01 mole) and respective ketone (0.01 mole) were taken in round bottom flask along with 15 ml of ethanol. The mixture was stirred for five minutes and slowly added morpholine (0.012 mole) at 50° C. with stirring for 10-15 minutes. Later, the reaction mixture was allowed to stir for five hours at room temperature and left in refrigerator over night. Collected the crystals by filtration under reduced pressure and washed with cold alcohol and recrystallized with ethanol (Gewald, K.; Schinke, E.; Böttcher, H Chem. Ber. 99: 94-100, 1966). These compounds can also be prepared by using microwave method as described here. Sulphur (0.01 moles), melanonitrile (0.01 moles), respective ketone (0.01 moles) and morpholine (0.012 moles) were taken in a sealed tube along with basic alumina (0.02 moles). The tube was irradiated with microwave energy at 160 watts for ten minutes. The tube was cooled and compound was extracted using ethyl acetate. The pure product was separated by performing column chromatography (Huang, W and Li, J, Synthetic Comm. 35: 1351-1357, 2005).

For title compounds, the 4,5-substituted 2-amino 3-cyano thiophenes (Ia-d) (0.0065 moles) was added to cyclic ketones (0.013 moles) in 1:2 ratio in a round bottomed flask along with zinc chloride (0.0065 moles) and heated the reaction mixture under reflux for five hours. Cooled the mixture and added to 50 ml of 40% sodium hydroxide solution in water to release the product from zinc chloride complex. Collected the separated precipitate using vacuum filtration and purified the product by passing through a column using a mixture of hexane and ethyl acetate as mobile phase (Ikuo, K.; Kijino, A.; Imai, A.; Yasumura, M.; (Hodogaya Chemical Co., Ltd., Japan), JKXXAF JP 04134083 (1992).

The above methods of synthesis are for purpose of illustration. The actual synthesis of either the precursor 2-amino, 3-cyano thiophenes may be carried out by other conventional techniques. Likewise the actual conversion of the 2-amino, 3-cyano thiophenes to the title compounds may also be carried out using conventional techniques.

### Tumour Necrosis Factor

Tumour necrosis factor is a polypeptide cytokine involved in inflammation and the acute phase response. TNF-α is present in larger quantities in persons with rheumatoid arthritis or Crohn's disease. Direct inhibition of TNF-α by the commercial biological agents etanercept (Enbrel), infliximab (Remicade), adalimumab (Humira), has produced significant advances in rheumatoid arthritis treatment and validated the extra-cellular inhibition of this proinflammatory cytokine as an effective therapy. However, despite considerable incentives, viable leads for analogous small-molecule inhibitors of TNF-α have not been reported. Such drugs with attendant advantages in manufacturing, patient accessibility, administration, and compliance would represent a major advance in the treatment of TNF-α mediated diseases. The immune system is often involved with inflammatory disorders, like both allergic reactions and some myopathies, with many immune system disorders resulting in abnormal inflammation. Non-immune diseases with etiological origins in inflammatory processes are thought to include cancer, atherosclerosis and ischemic heart disease which can be controlled by inhibiting the TNF-α.

Autoimmune disorders develop when the immune system destroys normal body tissues. Normally, the immune system is capable of differentiating “self” from “non-self” tissue. If some immune system cells (lymphocytes) become sensitized against “self” tissue cells, these cells are usually controlled by other lymphocytes. Autoimmune disorders occur when the control process is disrupted or when normal body tissue is altered so that it is no longer recognized as “self.” There are more than 80 types of autoimmune diseases and a person may experience more than one autoimmune disorder at the same time. Examples of autoimmune (or autoimmune-related) disorders include: rheumatoid arthritis, ankylosing spondylitis, ulcerative colitis, psoriasis and Crohn's disease, among others.

As part of the immune response, the body naturally produces the protein TNF-α to mobilize white blood cells to fight infections and other invaders. This response temporarily causes inflammation in the affected area. A person with an inflammatory condition is not able to remove TNF-α naturally, causing more and more white blood cells to travel to the affected area. As TNF-α continues to build up, it causes excessive inflammation, leading to pain and tissue damage.

α-Glucosidase and α-amylase

At present there are many pharmacological agents like sulphonylureas, Biguanides, Meglitinides, Glitazones, and α-Glucosidase Inhibitors are available for the management of diabetes.

One therapeutic approach for treating diabetes is to decrease the post-prandial hyperglycemia. This is done by retarding the absorption of glucose through the inhibition of the carbohydrate-hydrolysing enzymes such as α-glucosidase and α-amylase in the digestive tract. Inhibitors of these enzyme delay carbohydrate digestion and prolong overall carbohydrate digestion time, causing a reduction in the rate of glucose absorption and consequently blunting the postprandial plasma glucose rise.

Glucosidases are responsible for the catalytic cleavage of α-glycosidic bond in the digestive process of carbohydrates with specificity depending on the number of monosaccharides, the position of cleavage site, and the configuration of the hydroxyl groups in the substrate. α- and β-glucosidases are most extensively studied and are known to catalyze the hydrolysis of the glycosidic bonds involving a terminal glucose at the cleavage site. Of the two popular glucosidases, α-glucosidase (EC 3.2.1.20) has drawn a special interest of the pharmaceutical research community because it was shown that the inhibition of its catalytic activity led to the retardation of glucose absorption and the decrease in postprandial blood glucose level. This indicates that effective a -glucosidase inhibitors may serve as chemotherapeutic agents for clinic use in the treatment of diabetes and obesity. The catalytic role in digesting carbohydrate substrates also makes α-glucosidase a therapeutic target for the other carbohydrate-mediated diseases including cancer viral infections and hepatitis.

Slower digestion and absorption of carbohydrates reduces postprandial rise in plasma glucose. Acarbose has also been shown to increase secretion of glucagon-like peptide (GLP)-1, although relative contribution of this effect to the reduction in postprandial hyperglycemia is unknown. Both acarbose and miglitol are similarly effective in reducing postprandial hyperglycemia. In general, this class of drugs lowers HbA1c levels by about 0.5%. Many patients do not tolerate these agents due to flatulence, abdominal pain, and diarrhea.

Since the discovery of acarbose, that is the first member of α-glucosidase inhibitors approved for the treatment of type 2 diabetes, a variety of α-glucosidase inhibitors have been discovered as recently reviewed in an extensive fashion. These include transition state analogues (Lille et al., 2002) newly identified synthetic compounds and natural products isolated from a variety of species. The current research project is designed for the evaluation of the thienopyridine derivatives for antidiabetic activity by alpha-glucosidase and alpha-amylase inhibition.

### Significance of Carbohydrates

Carbohydrates are one of the very important classes of biomolecules. They have variety of function in the body. Generally, glucose serves as fuel to the cell it's stepwise oxidation yields ATP which provides energy for the cellular functions and polysaccharides (glycans) like starch and glycogen serve as stored fuel.

Polysaccharides and oligosaccharides are information carriers: they serve as destination labels for some proteins and as mediators of specific cell-cell interactions and interactions between cells and the extracellular matrix. Specific carbohydrate containing molecules act in cell-cell recognition and adhesion, cell migration during development, blood clotting, the immune response, and wound healing, to name but a few of their many roles. In most of these cases, the informational carbohydrate is covalently joined to a protein or a lipid to form a glycoconjugate, which is the biologically active molecule.

Dietary Carbohydrates Carbohydrate components present in a food can be characterized by: (i) their chemical identities, which are determined by botanical origin and in the case of composite foods the mix of ingredients used; (ii) the food matrix, which in addition to botanical origin is determined by the degree of processing during food manufacture and during food preparation. Together, these physico-chemical properties of the food largely determine the gastrointestinal handling and utilization of dietary carbohydrates.

Chemical identity of dietary carbohydrates is defined by sugar type, the linkages between sugars and the degree of polymerisation, which determines at the chemical level whether the endogenous digestive enzymes can hydrolyse the carbohydrate and in what form they are presented for metabolism.

Starch: Starch is major dietary Carbohydrate. In the plant, starch exists in the form of starch granules, which due to their comparatively small size survive milling relatively intact. This means that despite the disruption of the plant cell wall structures in raw flour preparations, the access of digestive enzymes is still restricted by the starch granule.

The form of the crystalline structure of starch granules is an important determinant of their digestibility, and is determined by botanical origin. Starch granules in potato and plantain (and green banana) are resistant to pancreatic amylase, digestibility of legumes is intermediary and cereal starch granules are typically more susceptible to digestion. Ratio of amylose: amylopectin can have an effect on starch digestibility, as amylose tends to form secondary structures that are hard to disperse, both in the starch granule and after food processing. However, for most foods influence of amylose: amylopectin is overshadowed by greater effect of food processing. Exceptions are very high-amylose starches, which can be difficult to disperse and resist digestion in the small intestine.

When heated in the presence of water, starch granules disrupt and gelatinize into a form easily available to pancreatic amylase while cooling does not reverse this process, some starch, especially high-amylose starches, may retrograde into forms less susceptible to digestion. Heating in the absence of water does not result in gelatinization, and starch granules remain intact in some types of dry baked foods, as with some biscuits, and the starch in such products is therefore digested slowly.

Carbohydrate and Glycemic Effect: The main component of diet that has the greatest influence on blood glucose is carbohydrate. Both quantity and type or source of carbohydrate found in food influences the post prandial blood glucose level. Total amount of carbohydrate consumed is a strong predictor of glycemic response. A variety of factor intrinsic to a given food can influence impact on blood glucose, including physical form of food, ripeness, degree of processing type of starch, style of preparation and time, amount of heat or moisture. Extrinsic variables such as, coingestion of protein and fat. Prior food intake, fasting glucose level, and insulin level will also alter the effect of specific carbohydrate containing food on blood glucose.

Postprandial Glycemic Response: Blood glucose concentration following a meal is determined by the rate of appearance of glucose in blood stream and its clearance from circulation. The rate of disappearance of glucose is largely influenced by the insulin secretion and its action on the target tissue.

In the post-absorptive state (on awakening in the morning) blood glucose is normal and stable, being derived from a constant production of endogenous glucose mainly by the liver matching the requirements of tissues. After breakfast, there is a new flux of glucose into the circulation, and gut transport and hepatic and extra-hepatic tissues maintain normal carbohydrate tolerance. In a non-diabetic individual, the liver minimizes post-prandial hyperglycemia both by increasing glucose uptake and by suppressing endogenous glucose production Thirty percent of the glucose intake is cleared by the liver and the rest reaches the peripheral circulation. Post-prandial suppression of endogenous glucose production can prevent 20-30 g of glucose from entering the systemic circulation.

Postprandial Hyperglycemia and Its Clinical Significance: Postprandial hyperglycemia is characterized by hyperglycemic spikes that induce endothelial dysfunction, inflammatory reactions and oxidative stress, which may lead to progression of atherosclerosis and occurrence of cardiovascular events.

Pathophysiology of postprandial hyperglycemia is characterized by hyperglycemic spikes that induce oxidative stress which in combination with soluble advanced glycation end products (AGEs) and lipid peroxidation products, act as key activators of upstream kinases, leading to endothelial dysfunction and expression of inflammatory genes.

Recent evidence suggests that almost 2 of 3 patients with symptomatic cardiovascular disease have abnormal glucose homeostasis. A significant number of these patients are not detected by increased fasting glucose levels, but rather by the presence of elevated glucose levels following a meal or during an oral glucose tolerance test. Postprandial hyperglycemia often occurs, even in the setting of good diabetic control assessed by hemoglobin A1c (HbA1c) and fasting glucose levels. In the early stages of type 2 diabetes, even when fasting glucose and HbA1c are within normal ranges, postprandial hyperglycemia causes macrovascular complications such as myocardial infarction or stroke as well as microvascular complications. Emerging data indicate that even impaired glucose tolerance may predispose to progression of atherosclerosis and cardiovascular events. There is evidence that postprandial hyperglycemia, but not fasting hyperglycemia, independently predicts the occurrence of cardiovascular events.

### Control of Postprandial Hyperglycemia (PPHG)

Fasting and postprandial plasma glucose concentrations, although due to different pathologic mechanisms, are interrelated. Higher the plasma glucose level with which a patient goes to bed as a result of postprandial hyperglycemia, higher will be fasting hyperglycemia in the morning. Similarly, higher the fasting hyperglycemia in the morning, the higher postprandial hyperglycemia will be during the day. Thus, maneuvers that primarily target fasting hyperglycemia might not be successful in normalizing fasting plasma glucose levels and achieving satisfactory HbA1c levels if postprandial hyperglycemia persists. Conversely, interventions that primarily target postprandial hyperglycemia might fail to achieve satisfactory HbA1c levels if fasting hyperglycemia persists. Regardless, it is believed that for patients with normal or near-normal fasting plasma glucose levels but elevated HbA1c levels, maneuvers that target postprandial hyperglycemia should deserve strong consideration as the initial choice.

The following examples are not to be construed as limiting the scope of the invention and are for the purpose of illustration.

Unless otherwise noted, chemicals were commercially available and used as received without further purification. All heating reactions were carried out on 12-place reaction station of Radleys Discovery Technologies. Thin layer chromatography was performed on precoated silica gel F₂₅₄ (Merck). Column chromatography was performed using silica gel 60-120 mesh (Merck). Melting points, were determined in open glass capillaries using a Polmon melting point apparatus and uncorrected. Infra red spectra recorded on Perkin-Elmer infrared spectrophotometer in KBR pellet. Mass spectra obtained on VG-7070H mass spectromotor. ¹H NMR spectra were recorded at 300 MHz on a Bruker Avance NMR spectrometer in CDCL₃ (δ 7.26) or DMSO-d₆ (δ 2.49).

### Example 1

### 2,3-dimethyl-6,7-dihydro-5H-cyclopenta[b]thieno[3,2-e]pyridin-4-amine (BN-1)

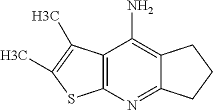

4,5-dimethyl-2-amino,3-cyano thiophene (0.0065 moles) was added to cyclopentanone (0.013 moles) in 1:2 ratio along with anhydrous zinc chloride (0.0065 moles) and the reaction mixture heated under reflux for five hours. The mixture was cooled and added to 50 ml of 40% NaOH solution in water to release the product from zinc chloride complex. The separated precipitate was collected using vacuum filtration and purified by passing through a column using a mixture of hexane and ethyl acetate as mobile phase.

Yield (%): 1.3 grams (90.9%); mp: 231° C., IR (KBr): 3505 cm⁻¹ (NH₂), ¹H NMR (CDCl₃, ppm): δ 2.12-2.20 (m, 2H, CH₂), 2.4 (s, 3H, CH₃), 2.5 (s, 3H, CH₃), 2.68-2.76 (t, 2H, CH₂), 2.95-3.05 (t, 2H, CH₂), 4.38 (s, 2H, NH₂, D₂O exchangeable), MS (ESIMS): m/z 219 [M⁺+1] 100%, Elemental Analysis: Calc. for C₁₂H₁₄N₂S: C, 66.02; H, 6.46; N, 12.83. Found: C, 66.32; H, 6.72; N, 12.66%.

### Example 2

### 2,3-dimethyl-5,6,7,8-tetrahydrothieno[2,3-b]quinolin-4-amine (BN-2)

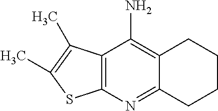

4,5-dimethyl-2-amino 3-cyano thiophene (0.0065 moles) was added to cyclohexanone (0.013 moles) in 1:2 ratio along with anhydrous zinc chloride (0.0065 moles) and the reaction mixture heated under reflux for 5 hours. The mixture was cooled and added to 50 ml of 40% NaOH solution in water to release product from zinc chloride complex. The separated precipitate was collected by vacuum filtration and purified by passing through a column using a mixture of hexane and ethyl acetate as mobile phase.

Yield: 1.45 grams (95.01%), mp: 210° C.; IR (KBr): 3504 cm⁻¹ (NH₂), NMR (CDCl₃, ppm): δ 1.82-1.90 (m, 4H, 2CH₂), 2.4 (s, 3H, CH₃), 2.45-2.47 (s, 2H, CH₂), 2.5 (s, 3H, CH₃), 2.95-3.15 (t, 2H, CH₂), 4.4 (s, 2H, NH₂, D₂O exchangeable); MS (ESIMS): m/z 233 [M⁺+1] 100% ; Elemental Analysis: Calc. for C₁₃H₁₆N₂S: C, 67.20; H, 6.94; N, 12.06. Found: C, 67.43; H, 6.78; N, 11.88%.

### Example 3

### 2,3,7-Trimethyl-5,6,7,8-tetrahydrothieno[2,3-b]quinolin-4-amine (BN-3)

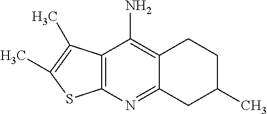

4,5-dimethyl-2-amino,3-cyano thiophene (0.0065 moles) was added to 3-methyl cyclohexanone (0.013 moles) in 1:2 ratio along with anhydrous zinc chloride (0.0065 moles) and the reaction mixture heated under reflux for 6 hours. The mixture was cooled and added to 50 ml of 40% NaOH solution in water to release product from zinc chloride complex. Separated precipitate was collected by vacuum filtration and purified by passing through a column using a mixture of hexane and ethyl acetate as mobile phase.

Yield (%): 1.52 grams (93.94%); mp: 220° C.; IR (KBr): 3502 cm⁻¹ (NH₂); ¹H NMR (CDCl₃, ppm): δ 1.44-1.55 (m, 3H, CH₃), 1.95-2.05 (m, 2H, CH₂), 2.4 (s, 3H, CH₃), 2.5 (s, 3H, CH₃), 2.55-2.60 (m, 5H, 2CH₂,CH), 4.25 (s, 2H, NH₂, D₂O exchangeable); MS (ESIMS): m/z 247 [M⁺+1] 100% ; Elemental Analysis: Calc. for C₁₄H₁₈N₂S: C, 68.25; H, 7.36; N, 11.37. Found: C, 68.08; H, 7.20; N, 11.62%.

### Example 4

### 2,3-dimethyl-6,7,8,9-tetrahydro-5H-cyclohepta[b]thieno[3,2-e]pyridin-4-amine (BN-4)

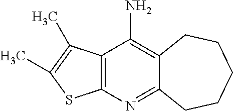

4,5-dimethyl-2-amino-3-cyano thiophene (0.0065 moles) was added to cycloheptanone (0.013 moles) in 1:2 ratio along with anhydrous zinc chloride (0.0065 moles) and the reaction mixture heated under reflux for six hours. The mixture was cooled and added to 50 ml of 40% sodium hydroxide solution in water to release the product from zinc chloride complex. The separated precipitate was collected by vacuum filtration and purified by passing through a column using a mixture of hexane and ethyl acetate as mobile phase.

Yield (%): 1.55 grams (95.79%); mp: 247.5° C.; IR (KBr): 3507 cm⁻¹ (NH₂); ¹H NMR (CDCl₃): δ 1.70-1.75 (m, 4H, 2CH₂), 1.82-1.90 (m, 2H, CH₂), 2.4 (s, 31-1, CH₃), 2.5 (s, 3H, CH₃), 2.62-2.68 (t, 2H, CH₂), 2.95-3.02 (t, 2H, CH₂), 4.5 (s, 2H, NH₂, D₂O exchangeable); MS (ESIMS): m/z 247 [M]⁺; Elemental Analysis: Calc. for C₁₄H₁₈N₂S: C, 68.25; H, 7.36; N, 11.37. Found: C, 68.50; H, 7.20; N, 11.54%.

### Example 5

### 2,3,6,7,8,9-Hexahydro-1H-benzo[4,5]thieno[2,3-b]cyclopenta[e]pyridin-10-amine (BN-5)

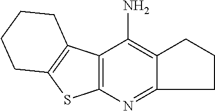

2-Amino-4,5,6,7-tetrahydrobenzo[b]thiophene-3-carbonitrile (0.0065 moles) was added to cyclopentanone (0.013 moles) in 1:2 ratio along with anhydrous zinc chloride (0.0065 moles) and the reaction mixture heated under reflux for six hours. The mixture was cooled and added to 50 ml of 40% sodium hydroxide solution in water to release the product from zinc chloride complex. The precipitate was collected by vacuum filtration and purified by passing through a column using a mixture of hexane and ethyl acetate as mobile phase.

Yield (%): 1.25 grams (91.24%); mp: 221° C.; IR (KBr): 3504 cm⁻¹ (NH₂); ¹H NMR (CDCl₃, ppm): δ 1.85-1.98 (m, 4H, 2CH₂), 2.12-2.22 (m, 2H, CH₂), 2.70-2.85 (m, 4H, 2CH₂), 2.96-3.08 (m, 4H, 2CH₂), 4.35 (s, 2H, NH₂, D₂O exchangeable); MS (ESIMS): m/z 245 [M⁺+1] 100% ; Elemental Analysis: Calc. for C₁₄H₁₆N₂S: C, 68.81; H, 6.60; N, 11.46. Found: C, 68.56; H, 6.43; N, 11.30%.

### Example 6

### 1,2,3,4,7,8,9,10-Octahydrobenzo[4,5]thieno[2,3-b]quinolin-11-amine (BN-6)

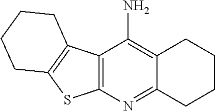

2-Amino-4,5,6,7-tetrahydrobenzo[b]thiophene-3-carbonitrile (0.0065 moles) was added to cyclohexanone (0.013 moles) in 1:2 ratio along with anhydrous zinc chloride (0.0065 moles) and the reaction mixture heated under reflux for six hours. The mixture was cooled and added to 50 ml of 40% sodium hydroxide solution in water to release the product from zinc chloride complex. The precipitate was collected by vacuum filtration and purified by passing through a column using a mixture of hexane and ethyl acetate as mobile phase.

Yield (%): 1.35 grams (93.16%); mp: 218.7° C.; IR (KBr): 3507 (NH₂), 3325, 3189, 2910, 2832 cm⁻¹; ¹H NMR (CDCl₃, ppm): δ 1.84-1.92 (m, 8H, 4-CH₂), 2.42-2.50 (m, 2H, CH₂), 2.82-2.92 (m, 4H, 2CH₂), 2.98-3.05 (t, 2H, CH₂) 4.44 (s, 2H, NH₂, D₂O exchangeable); MS (ESIMS):m/z 249 [M⁺+1] 100% ; Elemental Analysis: Calc. for C₁₅H₁₈N₂S: C, 69.73; H, 7.02; N, 10.84. Found: C, 69.91; H, 6.80; N, 10.59%.

### Example 7

### 8-Methyl-1,2,3,4,7,8,9,10-octahydrobenzo[4,5]thieno[2,3-b]quinolin-11-amine (BN-7)

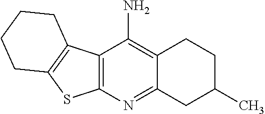

2-Amino-4,5,6,7-tetrahydrobenzo[b]thiophene-3-carbonitrile (0.0065 moles) was added to 3-methyl cyclohexanone (0.013 moles) in 1:2 ratio along with anhydrous zinc chloride (0.0065 moles) and reaction mixture heated under reflux for six hours. The mixture was cooled and added to 50 ml of 40% NaOH solution in water to release the product from zinc chloride complex. The precipitate was collected by vacuum filtration and purified by passing through a column using a mixture of hexane and ethyl acetate as mobile phase.

Yield (%): 1.40 grams (91.62%); mp: 207° C.; IR (KBr): 3503 cm⁻¹ (NH₂); ¹H NMR (CDCl₃, ppm): δ 1.50-1.55 (m, 3H, CH₃), 1.84-1.92 (m, 8H, 4CH₂), 1.95-2.05 (m, 2H, CH₂), 2.45-2.60 (m, 5H, 2CH₂,CH), 4.44 (s, 2H, NH₂, D₂O exchangeable);MS (ESIMS): m/z 273 [M⁺+1] 100% ; Elemental Analysis: Calc. for C₁₆H₂₀N₂S: C, 70.55; H, 7.40; N, 10.28. Found: C, 70.25; H, 7.66; N, 10.11%.

### Example 8

### 2,3,4,7,8,9,10,11-Octahydro-1H-benzo[4,5]thieno[2,3-b]cyclohepta [e]pyridin-12-amine (BN-8)

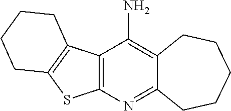

2-Amino-4,5,6,7-tetrahydrobenzo[b]thiophene-3-carbonitrile (0.0065 moles) was added to cycloheptanone (0.013 moles) in 1:2 ratio along with anhydrous zinc chloride (0.0065 moles) and the reaction mixture heated under reflux for six hours. The mixture was cooled and added to 50 ml of 40% sodium hydroxide solution in water to release the product from zinc chloride complex. The precipitate was collected by vacuum filtration and purified by passing through a column using a mixture of hexane and ethyl acetate as mobile phase.

Yield (%): 1.45 grams (95.39%); mp: 228° C.; IR (KBr): 3509 cm⁻¹ (NH₂); ¹H NMR (CDCl₃, ppm): δ 1.70-1.76 (4H, m, 2CH₂), 1.84-1.95 (m, 6H, 3CH₂), 2.60-2.65 (t, 2H, CH₂), 2.78-2.85 (t, 2H, CH₂), 2.98-3.08 (m, 4H, 2CH₂) 4.48 (s, 2H, NH₂, D₂O exchangeable); MS (ESIMS): m/z 273 [M⁺+1] 100% ; Elemental Analysis: Calc. for C₁₆H₂₀N₂S: C, 70.55; H, 7.40; N, 10.28. Found: C, 70.35; H, 7.65; N, 10.45%.

### Example 9

### 1,2,3,6,7,8-hexahydrocyclopenta [b]cyclopenta[4,5] thieno[3,2-e]pyridin-9-amine (BN-13)

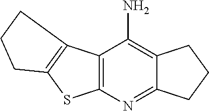

2-Amino-5,6-dihydro-4H-cyclopenta[b]thiophene-3-carbonitrile (0.0065 moles) was added to cyclopentanone (0.013 moles) in 1:2 ratio along with anhydrous zinc chloride (0.0065 moles) and the reaction mixture heated under reflux for six hours. The mixture was cooled and added to 50 ml of 40% sodium hydroxide solution in water to release the product from zinc chloride complex. The precipitate was collected by vacuum filtration and purified by passing through a column using a mixture of hexane and ethyl acetate as mobile phase.

Yield (%): 1.15 grams (82.02%); mp: 245° C.; IR (KBr): 3504 cm⁻¹ (NH₂), ¹H NMR (CDCl₃, ppm): δ 2.1-2.18 (2H, m, CH₂), 2.42-2.48 (m, 2H, CH₂), 2.69-2.72 (t, 2H, CH₂), 2.90-2.92 (m, 4H, 2CH₂), 3.02-3.04 (m, 2H, CH₂) 4.18 (s, 2H, NH₂, D₂O exchangeable); MS (ESIMS): m/z 233 [M⁺+1] 100%; Elemental Analysis: Calc. for C₁₃H₁₄N₂S: C, 67.79; H, 6.13; N, 12.16. Found: C, 68.05; H, 7.31; N, 11.91%.

### Example 10

### 2,3,6,7,8,9-Hexahydro-1H-cyclopenta[4,5]thieno[2,3-b]quinolin-10-amine (BN-14)

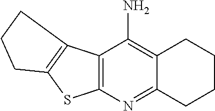

2-Amino-5,6-dihydro-4H-cyclopenta[b]thiophene-3-carbonitrile (0.0065 moles) was added to cyclohexanone (0.013 moles) in 1:2 ratio along with anhydrous zinc chloride (0.0065 moles) and reaction mixture heated under reflux for 6 hours. The mixture was cooled and added to 50 ml of 40% NaOH solution in water to release product from zinc chloride complex. Separated precipitate was collected by vacuum filtration and purified by passing through a column using a mixture of hexane and ethyl acetate as mobile phase.

Yield (%): 1.30 grams (87.42%), mp: 242° C.; IR (KBr): 3510 (NH₂), 3322, 3175, 2910, 2819 cm⁻¹; ¹H NMR (CDCl₃): δ 1.85-1.94 (m, 4H, 2CH₂), 2.44-2.55 (m, 4H, 2CH₂), 2.84-2.92 (t, 2H, CH₂), 2.93-3.02 (m, 2H, CH₂), 3.04-3.12 (m, 2H, CH₂), 4.28 (s, 2H, NH₂, D₂O exchangeable); MS (ESIMS): m/z 245[M]⁺; Elemental Analysis: Calc. for C₁₄H₁₆N₂S: C, 68.81; H, 6.60; N, 11.46. Found: C, 68.65; H, 6.83; N, 11.62%.

### Example 11

### 7-Methyl-2,3,6,7,8,9-hexahydro-1H-cyclopenta[4,5]thieno[2,3-b]quinolin-10-amine (BN-15)

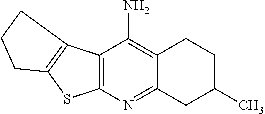

2-Amino-5,6-dihydro-4H-cyclopenta[b]thiophene-3-carbonitrile (0.0065 moles) was added to 3-methyl cyclohexanone (0.013 moles) in 1:2 ratio along with anhydrous zinc chloride (0.0065 moles) and reaction mixture heated under reflux for 6 hours. Mixture was cooled and added to 50 ml of 40% NaOH solution in water to release product from zinc chloride complex. Separated precipitate was collected by vacuum filtration and purified by passing through a column using a mixture of hexane and ethyl acetate as mobile phase.

Yield (%): 1.20 grams (76.28%); mp: 263.2° C.; IR (KBr): 3504 (NH₂), 3308, 3127, 2910, 2819 cm⁻¹, ¹H NMR (CDCl₃): δ 1.44-1.55 (m, 3H, CH₃), 1.95-2.05 (m, 2H, CH₂), 2.45-2.60 (m, 5H, 2CH₂,CH), 3.0-3.12 (m, 6H, 3CH₂), 4.25 (s, 2H, NH₂, D₂O exchangeable); MS (ESIMS): m/z 259[M]⁺; Elemental Analysis: Calc. for C₁₅H₁₈N₂S: C, 69.73; H, 7.02; N, 10.84. Found: C, 69.90; H, 7.28; N, 10.54%.

### Example 12

### 1,2,3,6,7,8,9,10-Octahydrocyclohepta[b]cyclopenta[4,5]thieno[3,2-e]pyridine-11-amine (BN-16)

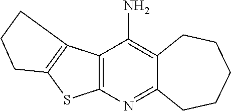

2-Amino-5,6-dihydro-4H-cyclopenta[b]thiophene-3-carbonitrile (0.0065 moles) was added to cycloheptanone (0.013 moles) in 1:2 ratio along with anhydrous zinc chloride (0.0065 moles) and reaction mixture heated under reflux for six hours. Mixture was cooled and added to 50 ml of 40% NaOH solution in water to release product from zinc chloride complex. The separated precipitate was collected by vacuum filtration and purified by passing through a column using a mixture of hexane and ethyl acetate as mobile phase.

Yield (%): 1.35 grams (85.82%); mp: 244.6° C.; IR (KBr): 3507 (NH₂), 3308, 3125, 2908, 2819 cm⁻¹; ¹H NMR (CDCl₃): δ 1.70-1.75 (m, 2H, CH₂), 1.80-1.92 (t, 2H, CH₂), 2.15-2.20 (t, 2H, CH₂), 2.48-2.55 (t, 2H, CH₂), 2.62-2.68 (t, 2H, CH₂), 2.95-3.05 (m, 4H, 2CH₂), 3.08-3.15 (m, 2H, CH₂), 4.30 (s, 2H, NH₂, D₂O exchangeable); MS (ESIMS): m/z 259[M]⁺; Elemental Analysis: Calc. for C₁₅H₁₈N₂S: C, 69.73; H, 7.02; N, 10.84. Found: C, 69.91; H, 7.24; N, 10.59%.

### Example 13

### 2,3,6,7,8,9,10,11-Octahydro-1H-cycloocta [b]cyclopenta[4,5]thieno[3,2-e]pyridin-12-amine (BN-17)

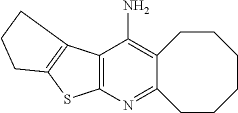

2-Amino-5,6-dihydro-4H-cyclopenta[b]thiophene-3-carbonitrile (0.0065 moles) was added to cyclooctanone (0.013 moles) in 1:2 ratio along with anhydrous zinc chloride (0.0065 moles) and heated under reflux for six hours. The mixture was cooled and added to 50 ml of 40% NaOH solution in water to release the product from zinc chloride complex, collected the separated precipitate using vacuum filtration and purified by passing through a column using a mixture of hexane and ethyl acetate as mobile phase.

Yield (%): 1.50 grams (90.44%); mp: 256° C.; IR (KBr): 3509 cm⁻¹ (NH₂); NMR (CDCl₃+DMSO-d₆, ppm): δ 1.70-1.74 (m, 2H, CH₂), 1.80-1.92 (m, 4H, 2CH₂), 2.15-2.20 (t, 2H, CH₂), 2.48-2.55 (t, 2H, CH₂), 2.62-2.68 (t, 2H, CH₂), 2.95-3.05 (m, 4H, 2CH₂), 3.08-3.15 (m, 2H, CH₂), 4.38 (s, 2H, NH₂, D₂O exchangeable); MS (ESIMS): m/z 273[M⁺+1] 100%; Elemental Analysis: Calc. for C₁₆H₂₀N₂S: C, 70.55; H, 7.40; N, 10.28; Found: C, 70.75; H, 7.24; N, 10.53%.

### Example 14

### 1,2,3,6,7,8,9,10-Octahydrocyclohepta[4,5] thieno[2,3-b]cyclopenta [e]pyridin-11-amine (BN-18)

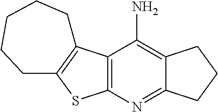

2-Amino-5,6,7,8-tetrahydro-4H-cyclohepta[b]thiophene-3-carbonitrile (0.0065 moles) was added to cyclopentanone (0.013 moles) in 1:2 ratio along with anhydrous zinc chloride (0.0065 moles) and heated under reflux for six hours. The mixture was cooled and added to 50 ml of 40% NaOH solution in water to release the product from zinc chloride complex. The separated precipitate was collected by vacuum filtration and purified by passing through a column using a mixture of hexane and ethyl acetate as mobile phase.

Yield (%): 1.20 grams (89.35%); mp: 255° C.; IR (KBr): 3509 cm⁻¹ (NH₂), ¹H NMR (CDCl₃, ppm): δ 1.70-1.75 (m, 2H, CH₂), 1.80-1.90 (t, 2H, CH₂), 2.15-2.20 (t, 2H, CH₂), 2.45-2.55 (t, 2H, CH₂), 2.62-2.68 (t, 2H, CH₂), 2.95-3.02 (m, 4H, 2CH₂), 3.08-3.15 (m, 2H, CH₂), 4.30 (s, 2H, NH₂, D₂O exchangeable); MS (ESIMS): m/z 259[M⁺+1] 100% ; Elemental Analysis Calc. for C₁₅H₁₈N₂S: C, 69.73; H, 7.02; N, 10.84. Found: C, 69.48; H, 7.24; N, 11.02%.

### Example 15

### 2,3,4,7,8,9,10,11-Octahydro-1H-cyclohepta[4,5]thieno[2,3-b]quinolin-12-Amine (BN-19)

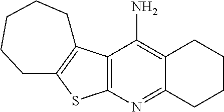

2-Amino-5,6,7,8-tetrahydro-4H-cyclohepta[b]thiophene-3-carbonitrile (0.0065 moles) was added to cyclohexanone (0.013 moles) in 1:2 ratio along with anhydrous zinc chloride (0.0065 moles) and heated the reaction mixture under reflux for six hours. Cooled the mixture and added to 50 ml of 40% NaOH solution in water to release the product from zinc chloride complex. Collected the separated precipitate using vacuum filtration and purified by passing through a column using a mixture of hexane and ethyl acetate as mobile phase.

Yield (%): 1.35 grams (95.74%); mp: 238° C.; IR (KBr): 3509 cm⁻¹ (NH₂), ¹H NMR (CDCl₃, ppm): δ 1.84-1.92 (m, 8H, 4-CH₂), 2.42-2.50 (m, 4H, 2CH₂), 2.82-2.92 (m, 4H, 2CH₂), 2.98-3.05 (t, 2H, CH₂) 4.25 (s, 2H, NH₂, D₂O exchangeable); MS (ESIMS): m/z 273[M⁺+1] 100% ; Elemental Analysis: Calc. for C₁₆H₂₀N₂S: C, 70.55; H, 7.40; N, 10.28; Found: C, 70.39; H, 7.63; N, 10.46%.

### Example 16

### Anti-Inflammatory Activity

### Test Animals:

Male Albino Wistar rats of 150-180 grams were used for the experiments. They were kept in polypropylene cages under standard laboratory conditions (12: 12 hr light/dark cycle at 24° C. Rats were provided with commercial rat diet (Pet Care, Bangalore) and water ad libitum and were divided into groups of six. The experiments were conducted after obtaining clearance from Animal Ethical Committee of IICT. All animals were quarantined and acclimatized to laboratory conditions for 7 days prior to study initiation. Animals were observed for general health and suitability for testing during this period.

### Methods:

### Carrageenan Induced Paw Edema Model

Anti-inflammatory activity of test compounds was evaluated in Wistar rats employing the method of Winter et al (1963) and Diwan et al (1989). Male Wistar rats were used for the study. Animals were fasted overnight and were divided into control, standard and different test groups each consisting of 3 animals. The different test compounds were administered to the animals in the test group at the dose of 100 mg/kg, by oral route. Animals in the standard group received Indomethacin at the dose of 10 mg/kg, by oral route. All test and standard compounds were administered as 1% gum acacia suspension. Rats in the control group received the vehicle solution without drugs. One hour after test drugs administration, rats in all the groups were challenged with 0.1 ml of 1% Carrageenan in the sub plantar region of right hind paw. A zero hour paw volume was measured for all the rats using digital plethysmometer (Ugo Basile, Italy) before the administration of carrageenan for all groups. Paw volumes were again measured 3 hrs after the challenge of Carrageenan. The percent inhibition of paw volume for each rat in treated groups was calculated by comparing with mean paw volume of control group and expressed as mean (±SE) percent inhibition of paw volume for each test. In a preliminary study the compounds that have shown good activity were evaluated for dose response study by using above procedure.

The synthetic compounds starting from BN-1 to BN-19 were evaluated for their anti inflammatory in acute model, carrageenan induced rat paw edema model. Some of these compounds in the series significantly (p<0.01 and p<0.05)) reduced the paw swelling (Table 1) comparing with control group. Among these compounds BN-4, BN-14 and BN-16 were found to be more active at the dose of 100 mg/kg. Based on preliminary results these compounds were evaluated for dose response study. Carrageenan induced edema is commonly used as an experimental animal model for acute inflammation and it is believed to be biphasic, of which the first phase is mediated by the release of histamine and 5-hydroxy tryptamine followed by kinin release and then prostaglandins in the later phase (Vinegar, 1969). Carrageenan induced hind paw edema is the standard experimental model of acute inflammation, and it is the phlogestic agent of choice for testing anti-inflammatory drugs. Moreover the experimental model exhibits high degree of reproducibility (Winter et al., 1962). In carrageenan induced paw edema model the three compounds have shown good anti-inflammatory potential in a dose dependant manner. The maximum effect was observed at the dose of 200 mg/Kg for BN-4, BN-14 and B-16 (53.2%, 56.45% and 57.53%) respectively and activity is comparable with standard drug Indomethacin at the dose of 10 mg/kg (p>0.05) as shown in the table 2. Based on the preliminary results the compounds BN-4, BN-14 and BN-16 were selected among all the compounds for further anti-inflammatory models.

[symbol]

[symbol]

| Compound | % Increase in Paw | |
|---|---|---|
| code | Volume | % Inhibition |
| Control-1 | 102.68 ± 3.14 | − |
| Control-2 | 119.57 ± 5.6 | − |
| Control-3 | 79.741 ± 0.38 | − |
| BN-1 | 87.92 ± 2.78# | 24.67 ± 5.49 |
| BN-2 a | 83.10 ± 2.66* | 20.64 ± 2.05 |
| BN-3 b | 81.44 ± 20.98# | 23.17 ± 5.15 |
| BN-4 b | 68.12 ± 3.41* | 41.79 ± 3.77 |
| BN-5 a | 100.6 ± 2.94 | 4.32 ± 1.19 |
| BN-6 a | 108.76 ± 2.73 | 3.14 ± 1.96 |
| BN-7 b | 113.12 ± 22.3 | 0.70 ± 0.70 |
| BN-8 a | 109.1 ± 4.87 | 1.20 ± 0.87 |
| BN-13 b | 91.74 ± 25.66* | 12.30 ± 1.95 |
| BN-14 b | 0.863 ± 0.0639 * | 33.47 ± 8.35 |
| BN-15 b | 86.54 ± 17.09* | 23.47 ± 8.98 |
| BN-16 b | 61.09 ± 19.16 * | 44.90 ± 5.86 |
| BN-17 c | 76.85 ± 6.45 | 6.87 ± 4.76 |
| BN-18 c | 62.84 ± 2.0 | 17.4 ± 0.27 |
| BN-19 c | 90.41 ± 11.36 | 0.00 ± 0.00 |
| Indomethacin | 41.69 ± 9.97* | 50.07 ± 12.07 |
| [symbol] | | |

[symbol]

indicates data missing or illegible when filed

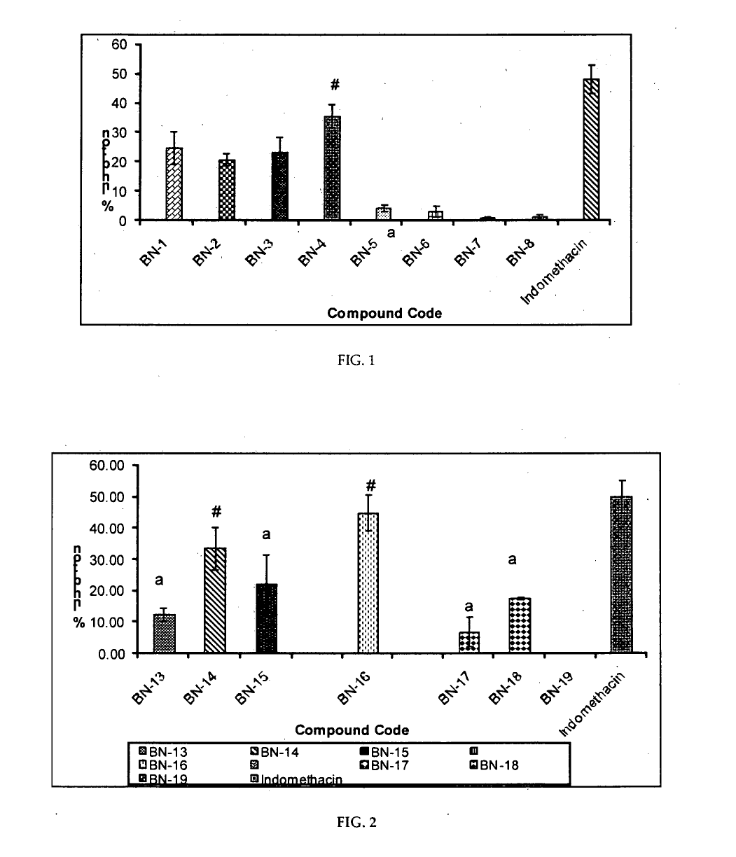

Table 1 shows percentage increase in paw volume and percentage inhibition for different Thienopyridine derivatives on carrageenan induced inflammation model in Wistar rats. The symbols * and # represents p<0.01 and p<0.05 comparing with respective control. Data is also presented in FIGS. 1, 2 and 3.

TABLE 2
Carrageenan Induced paw edema Model-Dose Response Study:

| | Percentage | | Percentage | | Percentage | |
|---|---|---|---|---|---|---|
| Compound | Swelling | % | Swelling | % | Swelling | % |
| Code | (50 mg/kg) | Inhibition | (100 mg/kg) | Inhibition | (200 mg/kg) | Inhibition |
| Control | 97.67 ± 4.25 | — | 119.57 ± 3.49 | — | 97.03 ± 2.15 | — |
| BN-4 | 73.27 ± 4.47* | 23.93 ± 2.96 | 64.42 ± 3.97* | 35.68 ± 3.77 | 44.23 ± 3.34* | 53.2 ± 3.03 |
| BN-14 | 72.34 ± 1.73* | 19.80 ± 2.24 | 71.30 ± 3.72* | 33.47 ± 6.70 | 41.78 ± 3.31* | 56.45 ± 3.12 |
| BN-16 | 73.18 ± 2.41* | 20.47 ± 1.61 | 61.09 ± 5.86* | 44.90 ± 5.86 | 45.60 ± 5.30* | 56.0 ± 2.87 |

Table 2 represents paw volumes and percentage of inhibition at different doses in dextran induced model. Symbol * represents the statistical significance difference (p<0.01) when compared with respective control. Values are expressed as Mean±S.E.M, (n=6).

### Example 17

### Dextran Induced Rat Paw Edema Model

Anti-inflammatory activity of test compounds was evaluated in Wistar rats employing method of Winter et al (1963) and Diwan et al (1989). Over night fasted rats were grouped into different test groups and standard group consisting 6 animals in each group. Test compounds were administered orally one hour before dextran challenge as gum acacia suspension. Animals in the standard group received Indomethacin at dose of 10 mg/kg. After one hour dextran was injected into the right hind paw by sub plantar route. A zero hour paw volume was measured for the rats using digital plethysmometer (Ugo Basile, Italy) before the administration of Dextran for all groups. Paw volumes were again measured 3 hrs after the challenge of Dextran. The percent inhibition of paw volume for each rat in treated groups was calculated by comparing with mean paw volume of control group and expressed as mean (±SE) percent inhibition of paw volume for each test group.

Compounds BN-4, BN-14 and BN-16 show good anti-inflammatory potential in a dose dependent manner. Oral administration of test compounds at dose of 100 and 200 mg/kg significantly suppressed paw volume (p<0.01). Compounds exhibited equal antiinflammaory potential (p>0.05) when compared with standard drug Indomethacin in Dextran induced paw edema model at dose of 200 mg/Kg. Dextran induced paw inflammation is mediated by both by histamine and Serotonin release from macrophages. Dextran induces fluid accumulation, which contains little protein and few neutrophills, where as carrageenan induces protein rich exudation containing large number of neutrophills (Kumar et al., 1995).

TABLE 3
Dextran induced paw edema-Dose response study

| | Percentage | | Percentage | | Percentage | |
|---|---|---|---|---|---|---|
| Compound | Swelling | % | Swelling | % | Swelling | % |
| Code | (50 mg/kg) | Inhibition | (100 mg/kg) | Inhibition | (200 mg/kg) | Inhibition |
| Control | 80.61 ± 5.94 | | 60.20 ± 2.25 | | 57.97 ± 2.96 | |
| BN-4 | 69.96 ± 4.06# | 12.17 ± 1.14 | 50.01 ± 1.09* | 13.57 ± 1.71 | 38.52 ± 2.60* | 32.71 ± 4.45 |
| BN-14 | 65.36 ± 2.91a | 21.29 ± 4.69 | 41.63 ± 2.78* | 24.83 ± 0.47 | 36.09 ± 2.79* | 38.54 ± 4.26 |
| BN-16 | 61.80 ± 1.18a | 18.30 ± 3.25 | 40.09 ± 4.24* | 30.90 ± 4.32 | 31.85 ± 2.09* | 41.00 ± 3.86 |

Table 3 represents paw volumes and percentage of inhibition at different doses in dextran induced model. * represents statistical significance difference (p<0.01) when compared with respective control. Symbol a indicates significant difference (p<0.05) compared with control. Values are expressed as Mean±S.E.M, (n=6).

### Example 18

### Arachidonic Acid Induced Paw Edema

Paw edema was produced in rats by Arachidonic acid following the method of Dimartino et al. (1987) and Laura segura et al., 1998. wistar rats weighing between 130-150 g were divided in to groups of six animals (n=6).A volume of 0.1 ml of 0.5% Arachidonic acid in 0.2M Carbonate buffer was injected in to the plantar side of the right hind paw of the rat. Paw volumes were measured using plethysmometer before and after one hour Arachidonic acid injection. Results obtained were compared with those obtained from the control group, which received vehicle only.

The compounds BN-4, BN-14 and BN-16 significantly reduced the percentage of swelling (p<0.01 and p<0.05) at different doses studied. The compounds have shown strong anti-inflammatory potential (44.92%, 41.37%, 45.63%) at the dose of 200 mg/kg in this model and activity is comparable with Dexamethasone at the dose of 3 mg/kg. It is known that in Arachidonic induced rat paw edema model Arachidonic acid derivatives, specially leukotriene, have an important role and COX inhibitors show low or no activity (Dimatrino. et. al; 1987). Since they possessed good antiinflammatory potential in Arachidonic induced model it is considered that they were inhibiting 5-lipoxygenase path way also. There is the hypothesis that dual inhibitors of Arachidonate, cyclooxygenase and Lipoxygenase may show more differential anti-inflammatory effect than selective cyclooxygenase inhibitors (Higgs et al., 1979; Higgs et al. 1981). Since the Lipoxygenase products are thought to be important mediators in anti inflammatory response as well as Cyclooxygenase products (Smith et al., 1980). BW755C (Higgs et al., 1979) and Timegadine (Ahnfelt-Ronne., 1980, Ahnfelt-Ronne., 1982) have been reported to be dual inhibitors of Arachidonic acid metabolism. In fact BW755C shows different effect from Indomethacin in reducing leukocytes migration in the rat carrageenan sponge model (Higgs et al., 1979; Higgs et al., 1983). In addition Benaxoprofen that is also a Lipoxygenase inhibitor with a weak inhibition of prostaglandin synthesis (Cashin et al., 1977) has been shown to produce greater effect than many NSAIDS at non-toxic doses in suppressing the bone damage in arthritic rats. Since the compounds were showing activity in Carragenan and Archidonic acid models, it can be postulated that these compounds may be acting in both COX and LOX pathways.

[symbol]

TABLE 4
Arachidonic acid Induced Paw Edema Model

| | Percentage | | Percentage | | Percentage | |
|---|---|---|---|---|---|---|
| Compound | Swelling | % | Swelling | % | Swelling | % |
| Code | (50 mg/kg) | Inhibition | (100 mg/kg) | Inhibition | (200 mg/kg) | Inhibition |
| Control | 68.05 ± 3.5 | | 70.36 ± 2.70 | | 68.41 ± 5.2 | |
| BN-4 | 55.32 ± 2.06# | 19.13 ± 2.01 | 48.54 ± 2.2* | 29.94 ± 3.37 | 37.50 ± 1.60* | 44.92 ± 3.10 |
| BN-14 | 50.56 ± 3.12* | 24.21 ± 3.89 | 45.64 ± 2.0* | 33.6 ± 3.11 | 38.70 ± 1.60* | 41.37 ± 2.40 |
| BN-16 | 52.32 ± 2.37* | 21.72 ± 2.80 | 44.95 ± 2.0* | 34.10 ± 2.82 | 36.30 ± 1.80* | 45.63 ± 2.87 |
| Dexamethason | [symbol] | [symbol] | [symbol] | [symbol] | [symbol] | [symbol] |

[symbol]

indicates data missing or illegible when filed

Table 4 represents paw volumes and percentage of inhibition at different doses in dextran induced model. * represents the statistical significance difference (p<0.01) when compared with respective control. The symbol # indicates significant difference (p<0.05) compared with control. Values are expressed as Mean±S.E.M, (n=6).

### Example 19

### Cotton Pellet Granuloma

### Sub Acute Model

Wistar rats weighing between 180-220 gms were anaesthetized with ether. The skin at their back was shaved and disinfected with 70% alcohol according to D'Arcy et al., 1960). An incision was made in the lumbar region. Using a blunt forceps subcutaneous tunnel was formed and a sterilized cotton pellet was placed on both sides in scapular region. Standard 20 mg cotton pellets was weighed and sterilized for the study. Rats were grouped into 5 groups of 6 animals each. Standard (Indomethacin) and test compounds (BB-4, BN-14, and BN-16) were administered daily for 7 consecutive days at dose of 100 mg/Kg and 5 mg/kg respectively. On 8^{th} day rats in all groups were sacrificed with anesthetic ether. Pellets were removed and kept overnight for incubation at 37° C. Then pellets were dried at 60° C. until they reach constant weight. The wet weight of pellets was also recorded after sacrificing. The average weights of pellets of control group and other treated groups were calculated. The percent change of granuloma weight was calculated for all the test groups comparing with control group.

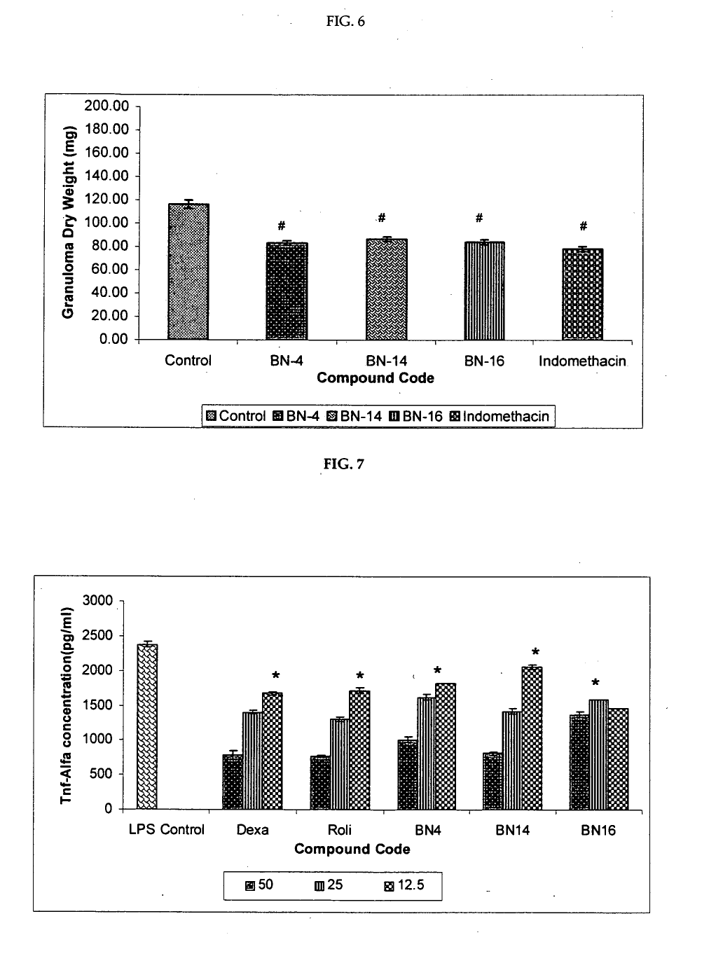

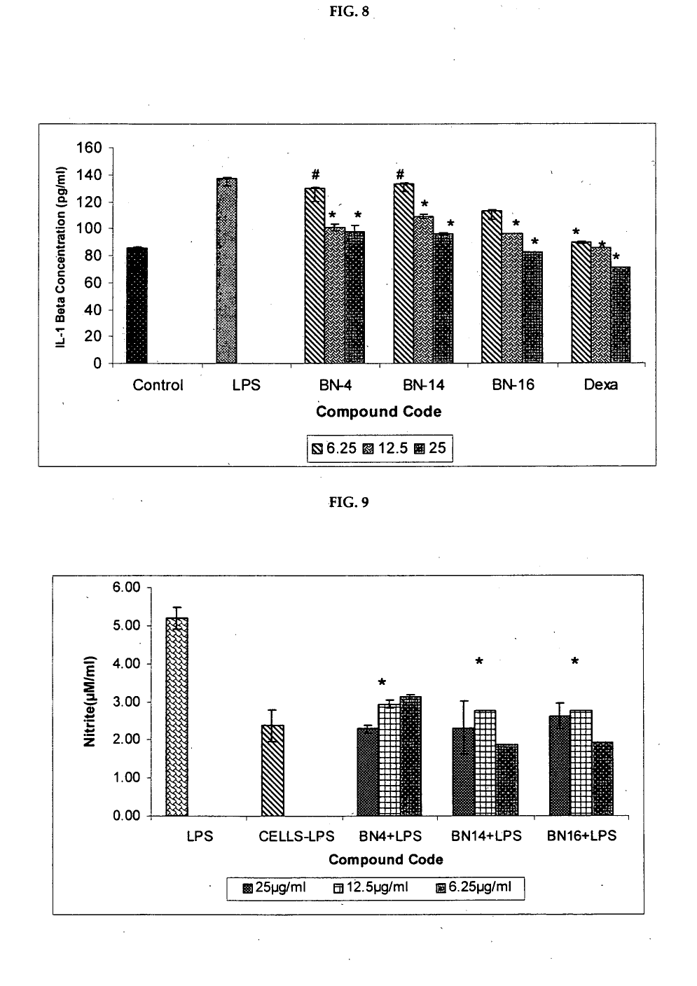

Compounds BN-4, BN-14 and BN-16 were evaluated for anti-inflammatory activity in subacute model, cotton pellet granuloma. This method has been widely employed to assess transudative, exudative and proliferative component of chronic inflammation and is a typical feature of established chronic inflammatory reaction. On 7th day of study implanted cotton pellets were removed and weighed and kept overnight in oven. Dry weight was recorded after drying for all groups. Both wet and dry granuloma weights significantly decreased comparing with control group (FIGS. 6 & 7).

Cotton pellet granuloma was significant inhibited (p<0.05) by test compounds and Indomethacin at doses of 100 mg/Kg and 5-mg/Kg respectively when compared with control. Cotton pellet granuloma inhibition was comparable with standard Indomethacin and there is no significant difference between test compounds and standard (P>0.05). This model is based on foreign body granuloma that is provoked in Wistar rats by subcutaneous implantation of pellets of compressed cotton. After several days histological giant cells and undifferentiated connective tissue can be observed beside fluid infiltration. Fluid absorbed by the pellet greatly influences wet weight of the granuloma and dry weight correlates well with the amount of granulation tissues formed (Spector W. G., 1979; Swingle K F., Shideeman F. E., 1972. Monocyte infiltration and fibroblast proliferation and exudation take place in chronic inflammation (Hosseinzadeh et al., 2000). This proliferation becomes widespread by proliferation of small vessels or granuloma (Hosseinzadeh et al., 2000). Nonsteroidal anti-inflammatory drugs decrease the size of granuloma which results from cellular reaction by inhibiting granulocyte infiltration/inflammation preventing generation of collagen and suppressing mucopolysaccharides (Lonac et al., 1996, Suleyman et al., 1999) fibers. During the study period no abnormal symptoms and mortality was observed in rat.

TABLE 5
effect of thienopyridine derivatives on cotton pellet granuloma study

| | | Granuloma | | Granuloma | |
|---|---|---|---|---|---|
| Compound | Dose | Wet Weight | | Dry | |
| Code | (mg/kg) | (mg) | % Inhibition | Weight (mg) | % Inhibition |
| Control | | 463.12 ± 12.52 | − | 116.42 ± 2.93 | − |
| BN-4 | 100 | 366.08 ± 3.50 | 22.23 ± 1.20* | 83.33 ± 3.59 | 28.42 ± 3.09* |
| BN-14 | 100 | 374.42 ± 4.69 | 20.21 ± 0.58* | 86.75 ± 2.20 | 25.48 ± 1.89* |
| BN-16 | 100 | 365.08 ± 6.39 | 23.09 ± 1.59* | 84.16 ± 2.11 | 27.70 ± 1.82* |
| Indomethacin | 5 | 338.01 ± 10.38 | 26.93 ± 2.24* | 78.25 ± 2.19 | 32.78 ± 1.88* |

Effect of Thienopyridine derivatives on cotton pellet granuloma study. Table shows cotton pellet weight and percent inhibition for compounds at dose of 100 mg/kg. * indicates no statistical difference compared to Indomethacin group. Values are expressed as Mean±S.E.M, (n=6).

### Example 20

### Ulcerogenic Assay

The method of Cashin et al., (1979) was employed. Food was withheld from the animals for 18 hours prior to the experiment. The fasted animals (n=4) received the compounds BN-4, BN-14 and BN-16 as gum acacia suspension by oral route at the dose of 100,200 mg/kg. Indomethacin control animals received the drug at the dose of 30 mg/kg. Six hours later, rats were killed and the stomachs removed and open along the lesser curvature. The opened stomach was washed with normal saline and observed for ulceration. Lesions on the mucosal surface was scored according to arbitrary scale: 0 No lesion; 0.5=hyperemia; 1=one or two lesions; 2=severe lesions; 3=very severe lesions; 4=Mucosal full of lesions (Bani et al., 2000). Oral administration of test compounds after 18 hr fasting did not produce ulceration at dose of 200 mg/kg in all rats (Table-6). But indomethacin group animals show severe ulceration in stomach portion. More importantly, gastric ulcer, which is a common side effect of NSAIDS, was totally absent in stomach of compound treated rat group. Since enhanced leukotrine production plays a role in development of gastrointestinal damage from cyclooxygenase inhibition by NSAIDS (Rainsford et al., 1993; Gyomber et al., 1996) and all the compounds have shown potent inhibitory action on 5-LOX inhibition (Arachidonic induced paw edema model) along with cox inhibition (Carrageenan induced paw edema model), the gastric ulcer sparing effect of compounds may be partly due to their effect on 5-LOX inhibition.

TABLE 6
Ulcerogenic Activity

| Compound | Dose | Ulcer | |
|---|---|---|---|
| Code | (mg/kg) | Incidence | Ulcer Score |
| CONTROL | − | 0/4 | 0.0 ± 0.0 |
| BN-4 | 200 | ¼ | 0.25 ± 0.12 |
| BN-14 | 200 | ¼ | 0.37 ± 0.10 |
| BN-16 | 200 | 0/4 | 0.25 ± 0.12 |
| Indomethacin | 30 | 4/4 | 2.25 ± 0.41 |

Table 6 shows ulcer index for the compounds BN-4,BN-14 and BN-16 at different doses. Lesions on the mucosal surface were scored according to an arbitary scale: 0=no lesions; 0.5=hyperemia; 1=one or two lesions; 2=severe lesions; 3=very severe lesions; 4=mucosa full of lesions.

### Example 21

### In Vitro TNF-α and No Estimation

Cell Culture and Treatment: The RAW 264.7 (mouse macrophages, national center for cellular sciences) cells were grown in plastic T-25 culture flask in Dulbeccos modified eagles medium (DMEM) without phenol red supplemented with 10% heat activated serum (heating at 57° C. for 30 minutes, 100 μg per ml penicillin, 200 μg per ml streptomycin, 2 mmol L-glutamine, 1% antibiotic/Anti mycotic solution (GIBCO/SRL) under 5% co2 with 85% relative humidity at 37° C. After 2-3 days cells were removed from the culture flask by tripsinizing the cells with Trypsin EDTA solution. Cell count and viability was performed using a standard Trypan blue dye exclusion method. The cell concentration was adjusted to 1×10⁵ cells per ml in each well with the same media in the 12 well plates. After 24 hrs incubation the media was removed from each well replaced with a fresh one containing different concentrations (50 μg, 25 μg, 12.5 μg) of the synthetic compounds BN-4, BN-14 and BN-16 along with 10 μg/ml LPS, where as normal control (PBS) and positive controls (Dexa, Roli) LPS (10 μg/ml), were added. 200 μl of media from each well was collected at 6 hrs for the estimation of TNF-α and after 24 hrs for Nitric Oxide respectively. The adherent cells, which remained after removing the media was subjected to MTD assay for determining the cytotoxicity of compounds at selected concentrations

Measurement of Nitrite: For determination of nitric oxide concentration, the stable conversion product of nitric oxide, nitrite (NO²⁻) was measured using the Griess reagent (Chi et, al., 2001). After 24 hrs incubation a volume of 100 μl of supernatant from each well of cell culture plates was transferred to 96 well micro plate and equal volume of Griess reagent (1% sulphanilamide, 0.1% N-(1-naphthyl ethylene diamine hydrochloride), 2.5% H₃PO₄) was then added to the supernatant at room temperature. The absorbance at 570 nm was determined in multi detection Microplate reader after 10 min. Nitric oxide concentration was calculated using standard curve.

Measurement of TNF-α: TNF-α was assayed by ELISA kit supplied by R&D systems. A volume of 100 μl of 0.8 μg per ml, goat anti mouse TNF-a in PBS was coated and incubated overnight at room temperature. Plates were washed 3 times with PBS/0.05% Tween 20 and blocked by the addition of 1% BSA in PBS at room temperature for 2 hrs. Plates were washed 3 times again with same buffer and 100 μl of samples and standards (Recombinant mouse TNF-α with 1% BSA in PBS) of different concentrations were added in triplicates and incubated for 2 hrs at RT. After washing 100 μl of goat anti mouse TNF-α conjugated with HRP were added and incubated for 2 hrs at RT. Then 100 μl of TMB substrate (Trimethyl benzidine with H2O2) was added to all wells. After 20 min stop solution was added to all the wells, and measured the optical density at 450 nm. TNF-α concentration was calculated from standard curve by regression analysis.

Nitric oxide inhibition Aaasy—Sodium Nitroprosside Method:

The procedure is based on the method, where sodium nitroprusside in aqueous solution at physiological PH spontaneously generates nitric oxide, which interacts with oxygen to produce nitrite ions that can be estimated using Griess reagent. For the experiment the reaction mixture containing sodium nitroprusside (20 mM) in phosphate buffer saline (pH 7.4), with or without the synthetic compounds at different concentrations, was incubated at 25° C. for 30 min in under a visible polychromatic light source (25 W tungsten lamp) (Marcocci et al., 1994). The reaction mixtures were mixed with an equal amount of Griess reagent (1% sulfanilamide in 5% phosphoric acid and 0.1% Naphthylethylenediamine dihydrochloride in water) (Green et al., 1982) and absorbance was measured at 570 nm. Inhibition of nitrite formation by the test compounds (BN-4, BN-14, BN-16) and the standard were calculated relative to the control.

To determine the mechanism of action all test compounds were screened for invitro anti-inflammatory methods, which include TNF-a and IL-1b and Nitric Oxide. All the three test compounds suppressed the release of proinflammatory cytokines from macrophage cell line at different concentrations (50 μg/ml, 25 μg/ml, 12.5 μg/ml). The compounds BN-4, BN-14, BN-16 have shown good inhibition in TNF-a and there was no significant statistical difference when compared with standards Dexamethasone and Rolipram at different concentrations (p>0.05). But BN-4 has not shown inhibition at the concentration of 12.5 μg/ml. TNF-a is a pleiotropic cytokine, which plays a critical role in both acute and chronic inflammation (Holtzmann et al., 2002). Several inflammagens have the ability of inducing the synthesis of TNF-a. Formation of a number of small molecular mediators of inflammation is linked with TNF-a and contributes to the range of mediators that critically control inflammation. TNF-a facilitates inflammatory cell infiltration by promoting the adhesion of neutrophils and lymphocytes to endothelial cells (Dinarello, 1997). When TNF-a is specifically blocked, severity of inflammation is reduced.

Effects of Thienopyridine Derivatives on Release of NO from Macrophage Cell Line:

Compounds BN-4, BN-14, BN-16 significantly (p<0.01) suppressed release of NO from murine macrophage culture cell line after 24 hrs additions of test compounds at different concentrations compared with LPS treated control sample (FIG. 10). NO is a highly fat soluble free radical, having numerous promiscuous roles. NO synthesis is amplified during inflammation. Several studies demonstrate that inflammation correlates with level of NO (Miller and Grisham, 1995). Since ROS (Reactive Oxygen Species) and RNS (Reactive Nitrogen species) generate mutations and cause DNA damage (Salgo, Bermudez; Squadrito, & Pryor, 1995). Murine Kacrophages can be stimulated by cytokines such as interleukin-1β, Interferon-γ or endotoxic (LPS), produced in large amounts of NO in vitro. Determination of cellular NO release is interesting since cytokine and endotoxin was found to induce over production of NO and its metabolites, in particular, peroxynitrite (Bloguh & Zafirioy, 1985). Both seem to contribute to numerous pathological conditions associated primarily with inflammation disorders (Clancy & Abramson, 1995). Therefore, murine macrophages like cellines, eg. RAW 264.7 and J744 are suitable cell models for performing invitro studies on the iNOs system. The Griess assay used to determine the NO level in LPS-treated macrophage has been found to be a suitable method for bio-guided fractionation of potential anti-inflammatory synthetic compounds.

### Scavenging Effect of Thienopyridine Derivatives on Sodium Nitroprusside Method:

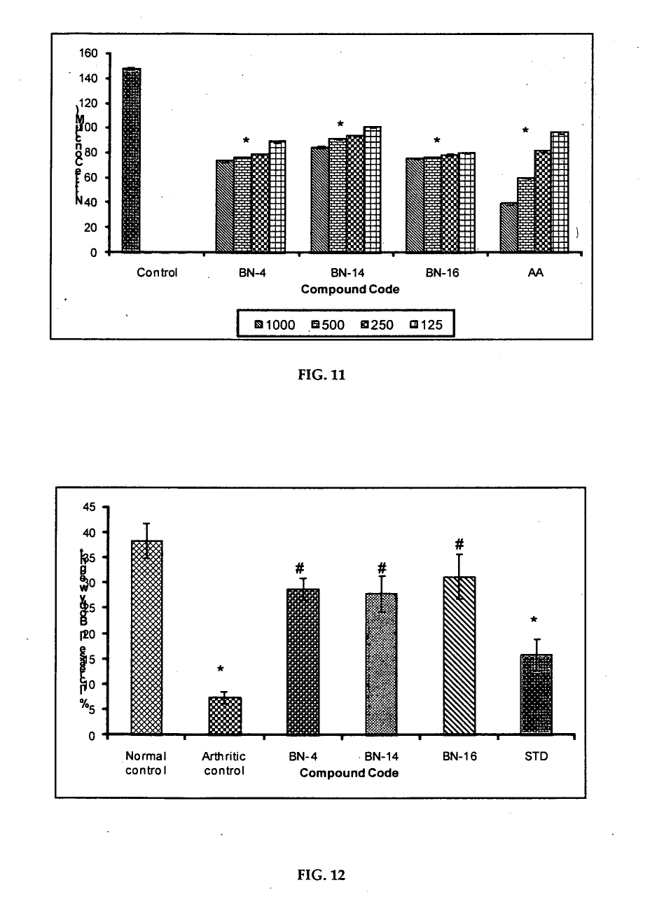

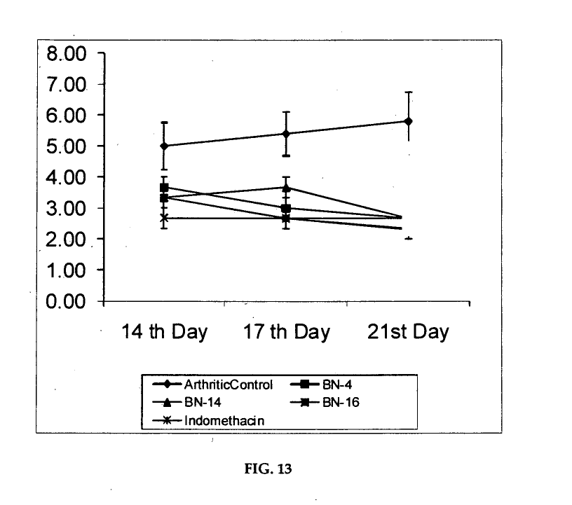

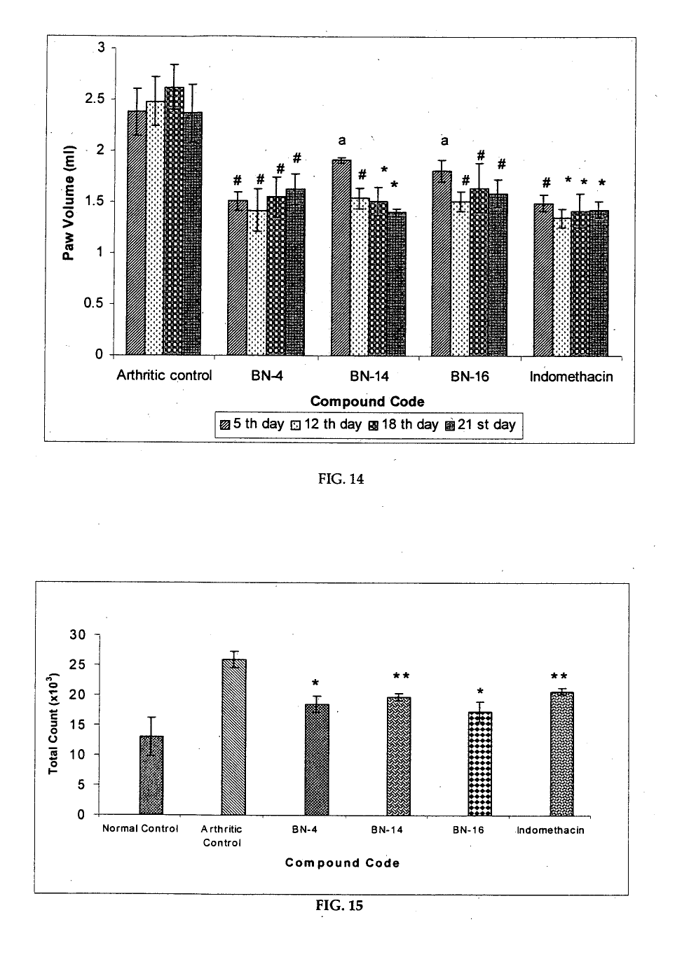

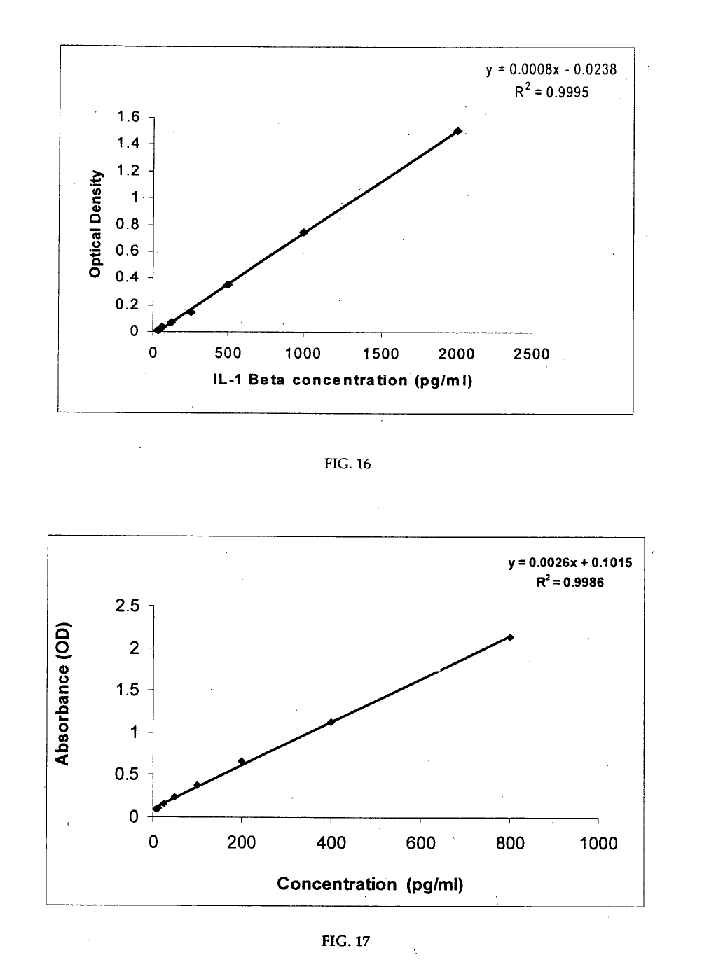

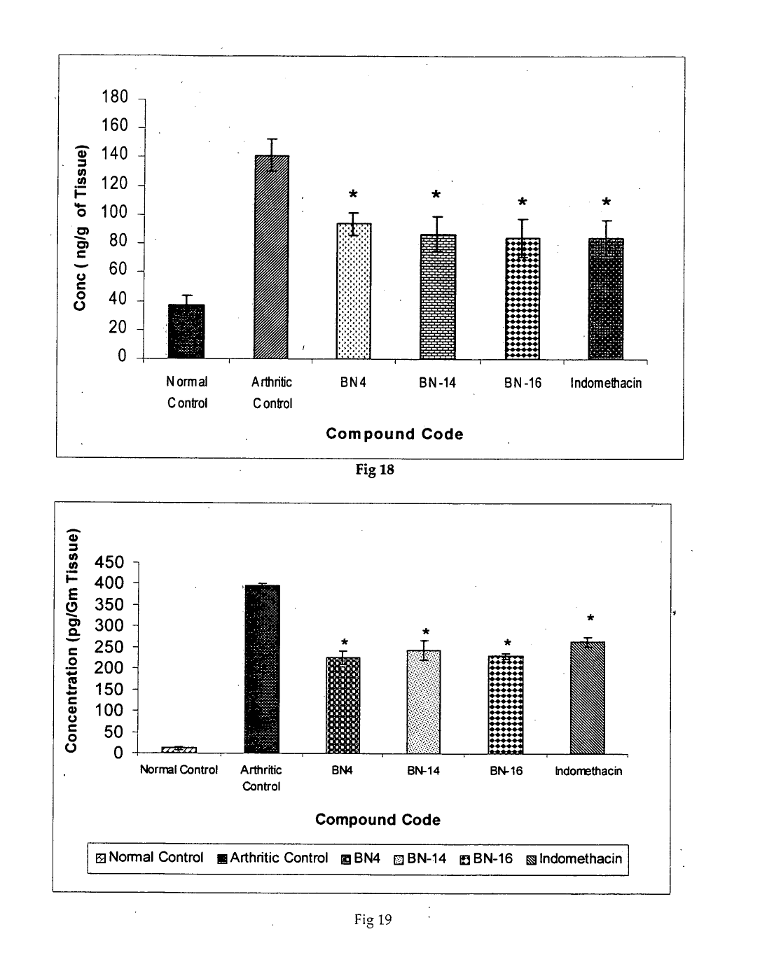

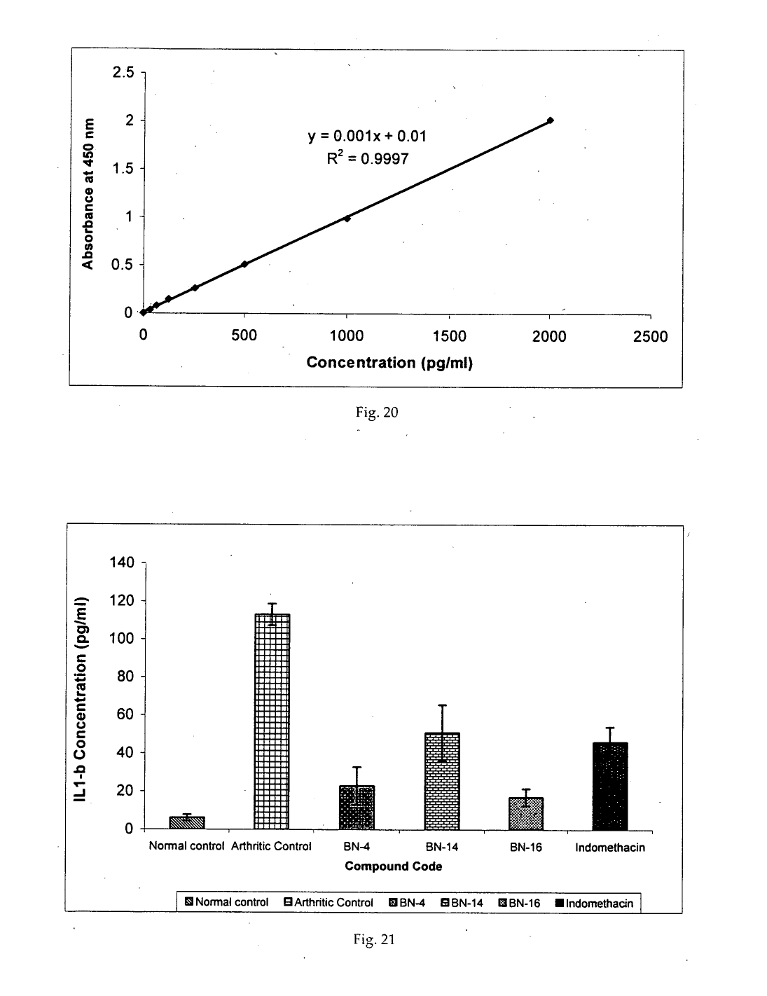

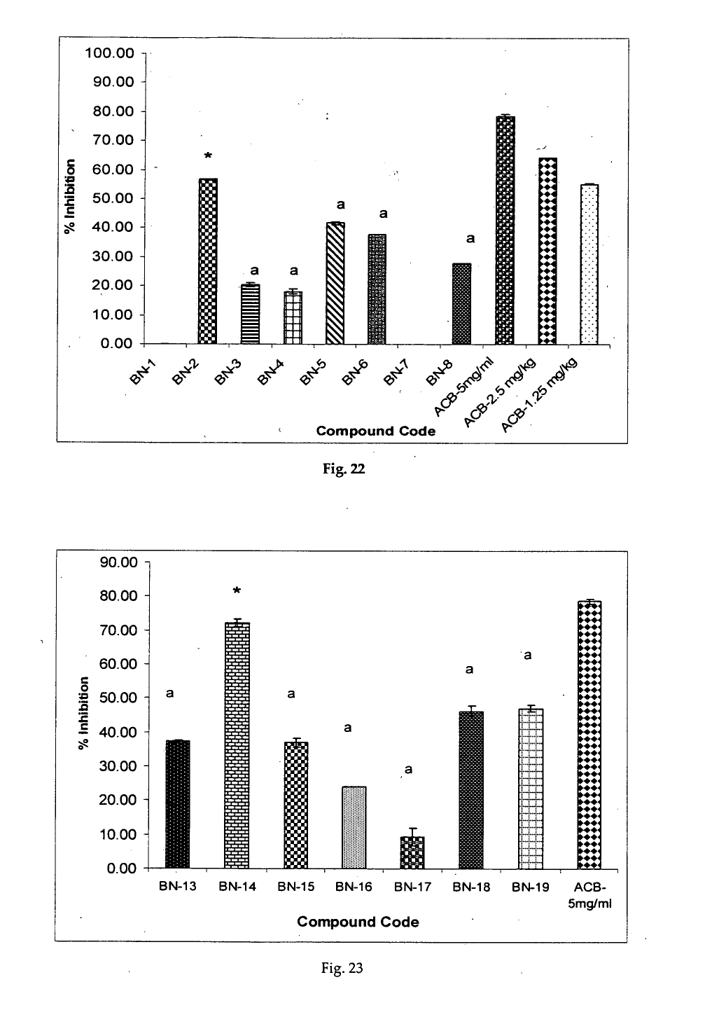

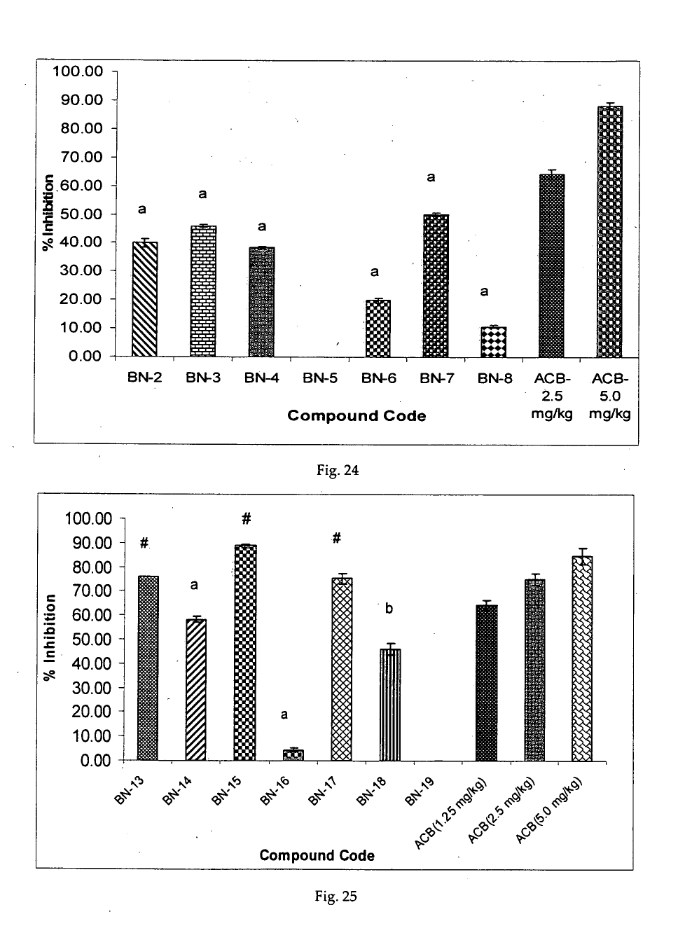

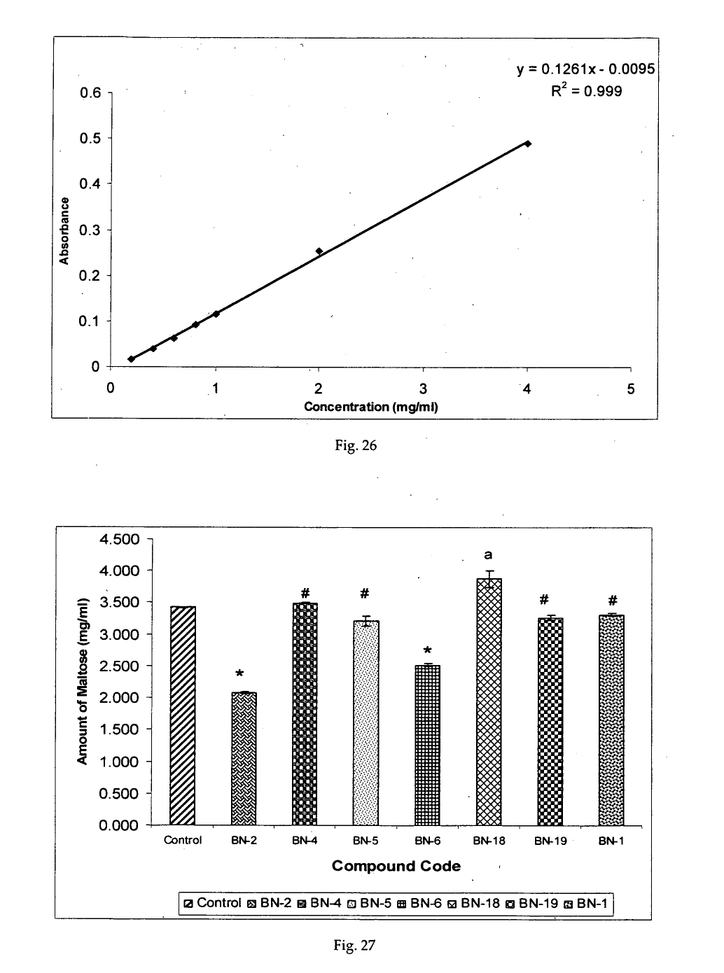

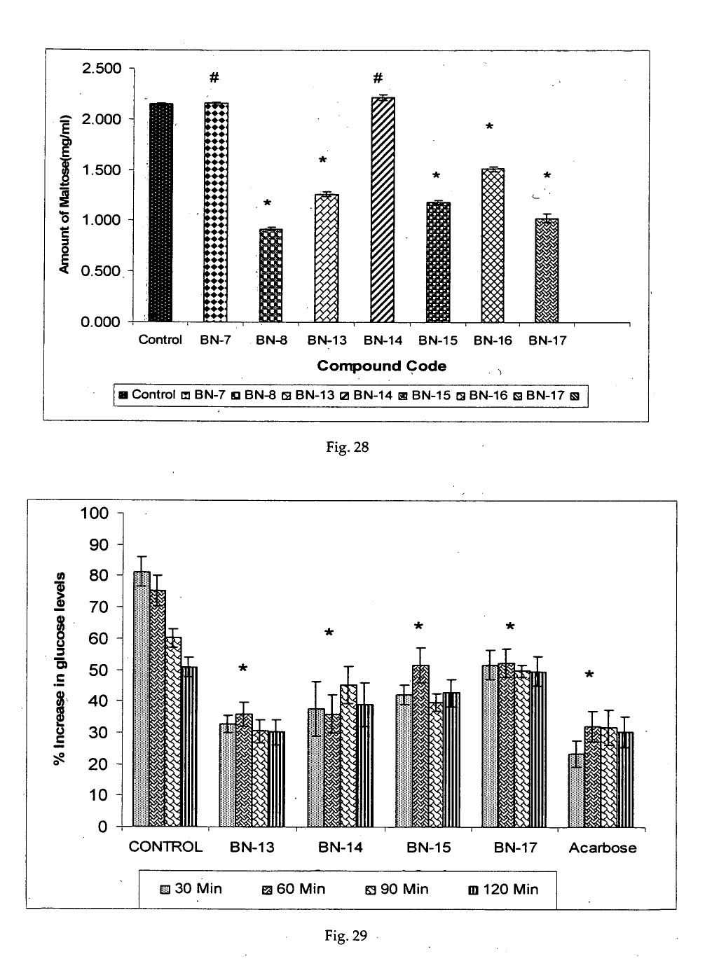

Compounds BN-4, BN-14, BN-16 significantly inhibited NO release in Sodium Nitro Prossuide method, as determined by Greiss reagent (FIG. 11). Stable NO products like NO₂, N₂O₄, N₃O₄ and NO₂ are reactive and are responsible for altering structural and functional behaviour of many cellular components. Incubation of solutions of Sodium Nitroprusside in phosphate buffer saline at 25° C. for 2 hrs resulted in linear time dependent Nitrite production, which is reduced by the tested synthetic compounds. It has become clear in recent years that NO acts as a part of the host defense mechanism. NO is generated in greatly increased amounts during infection and inflammation through immunological stimulation and shows cytotoxic or crytostatic activity against viruses and invasive organisms (ref). However, such as increase in NO may also have adverse effects on host cells, thus causing tissue injury (ref). For example, excess NO produced during ischemia reperfusion is considered to act as a toxic radical and to cause renal dysfunction, as does O₂ (ref). Antiinfective and anti-inflammatory reactions are achieved through inhibition of the formation of pro inflammatory mediators, such as prostaglandin's reactive oxygen species and NO.

### Example 22

### Anticancer Activity

A431 (Skin cancer), Colo-205 (Colon cancer), A549 (Lung cancer) cell lines were obtained from National Center for Cell science (NCCS), Pune, India. DMEM (Dulbeccos Modified Eagles Medium), MTT [3-(4,5-dimethylthiazol-2-yl)-2,5-diphenyl tetrazolium bromide], Trypsin, EDTA were purchased from Sigma Chemicals Co (St. Louis, Mo.), Fetal bovine serum were purchased from Arrow labs, 96 well flat bottom tissue culture plates were purchased from Tarson.

A431, Colo-205, A549 cell lines were grown as adherent cells in DMEM media supplemented with 10% fetal bovine serum, 100 μg/ml penicillin, 200 μg/ml streptomycin, 2 mM L-glutamine, and culture was maintained in a humidified atmosphere with 5% CO₂. Stock solution of 10 mg/ml was prepared for test molecules in DMSO. From the stock, various dilutions were made with sterile water to get required concentration. A431, Colo-205, A549 cell lines were seeded at a density of 1×10⁴ cells (cell number was determined by Trypan blue exclusion dye method) per each well in 100 μl of DMEM supplemented with 10% FBS. 12 hrs after seeding, above media was replaced with fresh DMEM supplemented with 10% FBS. Then, 10 μl sample from above stock solutions were added to each well in triplicates which gives final concentration of 100, 50, 25, 10 μg/well. The above cells were incubated for 48 hrs at 37° C. with 5% CO₂. After 48 hrs incubation the above media was replaced with 100 μl of fresh DMEM with out FBS and to this 10 μl of MTT (5 mg dissolved in 1 ml of PBS) was added and incubated for 3 hrs at 37° C. with 5% CO₂. After 3 hrs of incubation, the above media was removed with multi channel pipette, then 200 μl of DMSO was added to each well and incubated at 37° C. for 15 min. Finally, the plate was read at 570 nm using spectrophotometer (Spectra Max, Molecular devices). The results of the MTT assay using different cell lines were given below in table.

### Example 23

### Acute Toxicity Studies and Antiarthritic Activity

Freund's complete adjuvant (Difco), Indomethacin were purchased from Sigma Aldrich, USA .TNF-α and IL-1β kits from R & D Bio Systems were used.

Test Animals Male Albino Wistar rats of 150-180 Gms were used. They were kept in

| | A431 cell lines | Colo-205 | A549 |
|---|---|---|---|
| Compd | IC₅₀ (μg/ml) | IC₅₀ (μg/ml) | IC₅₀ (μg/ml) |
| BN-1 | 45.7 | 245 | 190 |
| BN-2 | 31.6 | 117 | 50.1 |
| BN-3 | 33.8 | 100 | 43.6 |
| BN-4 | 47.8 | No activity | 36.30 |
| BN-5 | 141 | No activity | 144.5 |
| BN-6 | 36.3 | 162 | 33.8 |
| BN-7 | 36.3 | 70.7 | 48.97 |
| BN-8 | 51.1 | 70.79 | 186 |
| BN-13 | 50.1 | 144 | 83.2 |
| BN-14 | 99.32 | 223 | 79.4 |
| BN-15 | 40.7 | No activity | 72.4 |
| BN-16 | 63.0 | 123 | 39.81 |
| BN-17 | 32.35 | 61.65 | 40.7 |
| BN-18 | 53.7 | 47.8 | 39.8 |
| BN-19 | − | − | − |

polypropylene cages under standard laboratory conditions (12: 12 hr light/dark cycle at 24° C., were provided with commercial rat diet (Pet Care, Bangalore) and water adlibitum. The rats were divided into 6 groups of 6 animals each. Experiments were conducted after obtaining clearance from Animal Ethical Committee of IICT. All animals were quarantined and acclimatized to laboratory conditions for 7 days prior to study initiation. Animals were observed for general health and suitability for testing during this period.

### Acute Oral Toxicity

In the series of thioenopyridine derivatives compounds BN-4, BN-14 and BN-16 show good antiinflammatory potential in all models tested. Based on these results the compounds were evaluated for safety by acute oral toxicity before proceeding to chronic antiarthritic screening. Acute Oral Toxicity Study was conducted following OECD guidelines by fixed dose method adopted by OECD (420). The study involved preliminary sighting using small number of animals in order to derive the dose effect relation for toxicity and mortality and to provide information on dose selection for the main study.

In the preliminary sighting study, the effect of various dosed administered to single animals of each sex was investigated in a sequential manner. The sighting study generally yields information on the dose-toxicity relationship including an estimate of the minimum lethal dose. In the main study, the test article is administered to groups of 5 males and 5 female animals at one of the fixed doses (5, 50, 500 and 2000 mg/Kg). The dose is derived from the sighting study. It is immediately below that which is expected to result in mortality. If evident toxicity is not seen at the chosen dose level, the substance should be retested at the next higher dose level. If at the initial dose level, animals die, or a severe toxicity reaction requires removal of animals from the study for animal welfare reasons, the substance is retested at the next lower dose level. Consideration of data from the both sighting study and the main study enabled a judgment to be made on determination of maximum tolerated dose.

Sighting Study: Animals were fasted overnight. Following period of fasting, the animals were weighed and were administered the test articles by oral route. After the test substance was administered food was withheld for further 3 to 4 hrs. In the sighting study, the effect of various doses was investigated in single animals of each sex. Dosing was sequential allowing at least 24 hrs before dosing the next animal. All animals were carefully observed for signs and symptoms of toxicity continuously up to 24 hrs and later up to 7 days. The sighting study was conducted with sequential doses of 5, 50, 500 and 2000 mg/kg of the test article. If the initial dose chosen did not produce severe toxicity, the next higher dose was selected. In this sighting study, the dose that produced evident toxicity but not death was identified. Dose escalation was continued up to 2000 mg/kg. The signs and symptoms of toxicity observed for each dose group have been tabulated in tables.

Test compounds were tested for toxicity. Compounds were administered as single dose by making suspension with gum acacia suspension after overnight fasting. Animals were observed for toxic symptoms for a period of one week. Then study was continued for 14 days to observe toxicity and mortality. Compounds BN-4 and BN-16 do not show any toxic symptoms and mortality at doses 5, 50, 500, and 2000 mg/kg throughout the study. Based on sighting study, dose 2000 mg/kg was selected for the main study in rats and mice for those compounds. Compound BN-14 was toxic at doses 2000 and 1250 mg/kg in rats and 2000 mg/kg in mice. A maximum dose 1060 mg/kg and 1250 mg/kg were selected respectively in rats and mice for the main study.

Acute Oral Toxicity of Thioenopyridine Derivatives
(Sighting Study in rats)

| Compound | | 5 | 50 | 500 | 2000 |
|---|---|---|---|---|---|
| Code | Observation | mg/kg | mg/kg | mg/kg | mg/kg |
| BN-4 | Bizarre physical position | X | X | X | X |
| | Bizarre tail position | X | X | X | X |
| | Muscle tremors | X | X | X | X |
| | Aggressiveness | X | X | X | X |
| | Inactivity | X | X | X | X |
| | Spontaneous Convulsions | X | X | X | X |
| | Dyspnea | X | X | X | X |
| | Sedation | X | X | X | X |
| | Nystagmus | X | X | X | X |
| | Cyanosis | X | X | X | X |
| | Abnormal excreta | X | X | X | X |
| | Salivation | X | X | X | X |
| | Nasal discharge | X | X | X | X |
| | Piloerection | X | X | X | X |
| | Phonation | X | X | X | X |
| | Death | X | X | X | X |
| | Altered muscle tone | X | X | X | X |
| | Catatonia | X | X | X | X |
| | Paralysis | X | X | X | X |
| | Change in pupil size | X | X | X | X |
| | Sensitivity to pain | X | X | X | X |
| | Skin lesions | X | X | X | X |
| | Corneal opacity | X | X | X | X |
| | Righting reflexes | X | X | X | X |
| | Bizarre physical position | X | X | X | X |
| | Bizarre tail position | X | X | X | X |
| | Muscle tremors | X | X | X | X |
| | Aggressiveness | X | X | X | X |
| | Inactivity | X | X | X | X |
| | Spontaneous Convulsions | X | X | X | X |
| | Dyspnea | X | X | X | X |
| | Sedation | X | X | X | X |
| | Nystagmus | X | X | X | X |
| | Cyanosis | X | X | X | X |
| | Abnormal excreta | X | X | X | X |
| | Salivation | X | X | X | X |
| | Nasal discharge | X | X | X | X |
| | Piloerection | X | X | X | X |
| | Phonation | X | X | X | X |
| | Death | X | X | X | X |
| | Altered muscle tone | X | X | X | X |
| | Catatonia | X | X | X | X |
| | Paralysis | X | X | X | X |
| | Change in pupil size | X | X | X | X |
| | Sensitivity to pain | X | X | X | X |
| | Skin lesions | X | X | X | X |
| | Corneal opacity | X | X | X | X |
| | Righting reflexes | X | X | X | X |
| BN-14 | Bizarre physical position | X | X | X | X |
| | Bizarre tail position | X | X | X | X |
| | Muscle tremors | X | X | X | X |
| | Aggressiveness | X | X | X | X |
| | Inactivity | X | X | X | X |
| | Spontaneous Convulsions | X | X | X | X |
| | Dyspnea | X | X | X | X |
| | Sedation | X | X | X | X |
| | Nystagmus | X | X | X | X |
| | Cyanosis | X | X | X | X |
| | Abnormal excreta | X | X | X | X |
| | Salivation | X | X | X | X |
| | Nasal discharge | X | X | X | X |
| | Piloerection | X | X | X | X |
| | Phonation | X | X | X | X |
| | Death | X | X | X | X |
| | Altered muscle tone | X | X | X | X |
| | Catatonia | X | X | X | X |
| | Paralysis | X | X | X | X |
| | Change in pupil | X | X | X | X |
| | Sensitivity to pain | X | X | X | X |
| | Skin lesions | X | X | X | X |
| | Corneal opacity | X | X | X | X |
| | Righting reflexes | X | X | X | X |
| | Bizarre physical position | X | X | X | X |
| | Bizarre tail position | X | X | X | X |
| | Muscle tremors | X | X | X | X |
| | Aggressiveness | X | X | X | X |
| | Inactivity | X | X | X | X |
| | Spontaneous Convulsions | X | X | X | X |
| | Dyspnea | X | X | X | X |
| | Sedation | X | X | X | X |
| | Nystagmus | X | X | X | X |
| | Cyanosis | X | X | X | X |
| | Abnormal excreta | X | X | X | X |
| | Salivation | X | X | X | X |
| | Nasal discharge | X | X | X | X |
| | Piloerection | X | X | X | X |
| | Phonation | X | X | X | X |
| | Death | X | X | X | X |
| | Altered muscle tone | X | X | X | X |
| | Catatonia | X | X | X | X |
| | Paralysis | X | X | X | X |
| | Change in pupil size | X | X | X | X |
| | Sensitivity to pain | X | X | X | X |
| | Skin lesions | X | X | X | X |
| | Corneal opacity | X | X | X | X |
| | Righting reflexes | X | X | X | X |
| BN-16 | Bizarre physical position | X | X | X | X |
| | Bizarre tail position | X | X | X | X |
| | Muscle tremors | X | X | X | X |
| | Aggressiveness | X | X | X | X |
| | Inactivity | X | X | X | X |
| | Spontaneous Convulsions | X | X | X | X |
| | Dyspnea | X | X | X | X |
| | Sedation | X | X | X | X |
| | Nystagmus | X | X | X | X |
| | Cyanosis | X | X | X | X |
| | Abnormal excreta | X | X | X | X |
| | Salivation | X | X | X | X |
| | Nasal discharge | X | X | X | X |
| | Piloerection | X | X | X | X |
| | Phonation | X | X | X | X |
| | Death | X | X | X | X |
| | Altered muscle tone | X | X | X | X |
| | Catatonia | X | X | X | X |
| | Paralysis | X | X | X | X |
| | Change in pupil size | X | X | X | X |
| | Sensitivity to pain | X | X | X | X |
| | Skin lesions | X | X | X | X |
| | Corneal opacity | X | X | X | X |
| | Righting reflexes | X | X | X | X |
| | Bizarre physical position | X | X | X | X |
| | Bizarre tail position | X | X | X | X |
| | Muscle tremors | X | X | X | X |
| | Aggressiveness | X | X | X | X |
| | Inactivity | X | X | X | X |
| | Spontaneous Convulsions | X | X | X | X |
| | Dyspnea | X | X | X | X |
| | Sedation | X | X | X | X |
| | Nystagmus | X | X | X | X |
| | Cyanosis | X | X | X | X |
| | Abnormal excreta | X | X | X | X |
| | Salivation | X | X | X | X |
| | Nasal discharge | X | X | X | X |
| | Piloerection | X | X | X | X |
| | Phonation | X | X | X | X |
| | Death | X | X | X | X |
| | Altered muscle tone | X | X | X | X |
| | Catatonia | X | X | X | X |
| | Paralysis | X | X | X | X |
| | Change in pupil size | X | X | X | X |
| | Sensitivity to pain | X | X | X | X |
| | Skin lesions | X | X | X | X |
| | Corneal opacity | X | X | X | X |
| | Righting reflexes | X | X | X | X |

### Main Study

At least 10 animals (5 males and 5 females) for each species were used for dose level in this study. Animals were fasted overnight and test articles were administered as 1% gum acacia suspension orally. The dose used in this study is selected from one of four levels 5 mg/kg, 50 mg/kg, 500 mg/kg and 2000 mg/kg i.e. the dose that produced evident toxicity but not mortality in sighting study. The animals were observed for following signs and symptoms of toxicity apart from cage side observations which include changes in skin and fur, eyes, mucous membrane, respiratory, circulatory, and autonomic and central nervous systems. The animals were observed for toxicity symptoms for a period of 14 days in each species and observations are tabulated. Different biochemical and hematology parameters observed on “0’ day and on termination day .During main study physical parameters like feed consumption and body weight increase were also recorded. 5 males and females were taken, and toxicity study conducted for the compounds. Compounds BN-4, BN-16 were found to be safe at dose of 2000 mg/kg and did not show any toxic symptoms. BN-14 was not toxic at dose 1060 mg/kg in rats and 1250 mg/kg in mice .No abnormal behavior was observed in both rats and mice during the study. No significant changes were observed in all the groups of rats and mice (BN-4, BN-14, and BN-16) in the main study. During the study physical parameters body weight was measured and uniform increase in body weight was observed in all compound groups of animals. Hematology and biochemical parameters were well and within normal range when compared with 0 day in rats and mice. Based on above observations synthetic compounds were safe upto 2000 mg/kg. The maximum tolerated dose was found to be beyond 2000 mg/kg for BN-4 and BN-16 in both rats and mice. Compound BN-14 showed toxic symptoms at doses 2000, 1250 mg/kg in rats and 2000 mg/kg in the mice. Maximum tolerated dose for BN-14 was found to be 1060 mg/kg in rats' and 1250 mg/kg in mice.

Acute Oral Toxicity of Thioenopyridine Derivatives
((Main Study in Rats)

| | | BN-4 | BN-14 | BN-16 |
|---|---|---|---|---|
| | Observation | 2000 mg/kg | 1060 mg/kg | 500 mg/kg |
| No Of | | | | |
| Animals | | | | |
| 5 M + 5 F | Bizarre physical | X | X | X |
| | position | | | |
| | Bizarre tail position | X | X | X |
| | Muscle tremors | X | X | X |
| | Aggressiveness | X | X | X |
| | Inactivity | X | X | X |
| | Spontaneous | X | X | X |
| | Convulsions | | | |
| | Dyspnea | X | X | X |
| | Sedation | X | X | X |
| | Nystagmus | X | X | X |
| | Cyanosis | X | X | X |
| | Abnormal excreta | X | X | X |
| | Salivation | X | X | X |
| | Nasal discharge | X | X | X |
| | Piloerection | X | X | X |
| | Phonation | X | X | X |
| | Death | X | X | X |
| | Altered muscle tone | X | X | X |
| | Catatonia | X | X | X |
| | Paralysis | X | X | X |
| | Change in pupil size | X | X | X |
| | Sensitivity to pain | X | X | X |
| | Skin lesions | X | X | X |
| | Corneal opacity | X | X | X |
| | Righting reflexes | X | X | X |
| Compound | | | | |
| Code | | | | |
| 5 M + 5 F | Bizarre physical | X | X | X |
| | position | | | |
| | Bizarre tail position | X | X | X |
| | Muscle tremors | X | X | X |
| | Aggressiveness | X | X | X |
| | Inactivity | X | X | X |
| | Spontaneous | X | X | X |
| | Convulsions | | | |
| | Dyspnea | X | X | X |
| | Sedation | X | X | X |
| | Nystagmus | X | X | X |
| | Cyanosis | X | X | X |
| | Abnormal excreta | X | X | X |
| | Salivation | X | X | X |
| | Nasal discharge | X | X | X |
| | Piloerection | X | X | X |
| | Phonation | X | X | X |
| | Death | X | X | X |
| | Altered muscle tone | X | X | X |
| | Catatonia | X | X | X |
| | Paralysis | X | X | X |
| | Change in pupil size | X | X | X |
| | Sensitivity to pain | X | X | X |
| | Skin lesions | X | X | X |
| | Corneal opacity | X | X | X |
| | Righting reflexes | X | X | X |

### Antiarthritic Effect of Thioenopyridines in Both FCA Induced Arthritis Studies

Freund's complete adjuvant (FCA) induced arthritis is a model extensively used to study pathogenesis of rheumatoid arthritis for testing therapeutics (Mizushima et al., 1972).

Induction of Arthritis: Arthritis was induced by injecting Freund's complete adjuvant (FCA. Required quantity of FCA was weighed and suspension made with paraffin oil (DIFCO Laboratories) and administered at dose of 0.5 mg/rat in subplantar region of the right hind paw. After 7 days, a booster injection was given to all animals to hind paws.

Experimental Design Animals were divided into 6 groups of 6 rats each and were designated as:

Group I—Normal Control; Group II—Arthritic control; Group III—Arthritic rats administered with Synthetic compound BN-4; Group IV—arthritic rats administered with compound BN-14; Group V—arthritic rats administered with BN-16; Group VI—arthritic rats administered with standard drug indomethacin. Administration of test compounds and indomethacin started on the same day of adjuvant injection at the dose of 100 and 2.5 mg/kg respectively. The test and indomethacin doses continued up to 21 days in FCA induced model and 24 days in collagen induced model. During the study period the paw volumes were measured on 5th, 12th, 18th and 21 st days and the weight of the animals was also recorded at different time intervals. At each time point the percentage of inhibition of paw volume was calculated with reference to mean paw volume of control group In FCA model, animals were sacrificed on 21^{st} day by cervical dislocation. Before sacrificing the animals, blood was collected and used for estimation of total leukocyte count, TNF-α and IL-1β.

Clinical Assessment of Adjuvant arthritis: Rats were examined for visual appearance of arthritis and the lesion of erythema and inflammation of periarticular tissues on 14^{th} day, 18^{th} day and on 21^{st} day. Arthritis scoring scale as follows:

0=No change

### 1=Mild Inflammation

2=Mild swelling and erythema of the limb
3=Gross swelling and erythema of the limb
4=Gross deformity and inability to use the limb

The arthritis score of each rat was the sum of the scores of both hind limbs and maximum score was being 8 for each rat.

Body weight Index: During the arthritic study changes in body weight have also been used to assess the course of disease and the response to therapy of anti-inflammatory drugs (Winder et al., 1969). As the incidence and severity of arthritis increased, the changes in the body weights of rats also reduced during the course of the experimental period. All test groups of rats gained significant increase (p<0.01) in body weight and the increase is comparable with Normal control group. Significant decrease in body weight was observed in arthritic group of rats in the FCA arthritis studies.

### Body Weight Index —FCA Study

| Compound Code | 1^{st} DAY | 21^{st} DAY |
|---|---|---|
| Normal Control | 155.7 ± 2.23 | 215.50 ± 2.82 |
| Arthritic Control | 159.88 ± 4.29 | 171.63 ± 4.16 |
| BN-4 | 155.15 ± 3.1 | 199.87 ± 4.76 |
| BN-14 | 158.02 ± 2.51 | 201.51 ± 6.61 |
| BN-16 | 157.85 ± 2.08 | 206.38 ± 4.76 |
| Indomethacin | 157.85 ± 2.08 | 182.68 ± 4.91 |

Arthritic Scoring: Arthritic scoring was done on 14th 17th and 21st day. Using arbitrary scale, scoring was performed for different groups and compared each time interval with arthritic control group. Arthritic scoring was significantly reduced (P<0.05; P<0.01) for all test compounds BN-4, BN-14 and BN-16 on 17th day and 21st day of study. Progression of disease was indicated by increased edema and erythema of one or both ankle joints, followed by involvement of metatarsal and interphalangeal joints. Fully developed arthritis including red and swollen paws was 8 to 10 days after onset of inflammation. Clinical score in arthritic control group reached approximately 14 days after immunization of FCA.

Paw volume Measurement: In arthritic study, paw swelling is one of the major factors assessing the degree of inflammation and therapeutic efficacy of the drugs (Begum and Sadique., 1988)). In the present study rats were selected to induce arthritis because rats develop a chronic swelling in multiple joints with influence of inflammatory cells, erosion of joint cartilage and bone destruction. It has close similarities to human rheumatoid arthritis disease (Singh and Majumdar., 1996) The determination of paw swelling is apparently simple, sensitive and quick procedure for evaluating the degree of inflammation and therapeutic effects of drugs. Chronic inflammation involves accumulation of macrophages, release of inflammatory mediators like cytokines (TNF-α, IL-1β, GM-CSF, interferon's and Platelet Growth Differentiation Factor (PGDF). These mediators are responsible for the pain, destruction of bone and cartilage that can lead to severe disability (Laurence, 1996).

Healthy Wistar rats were selected for the study. Adjuvant injection resulted in progressive swelling of injected paw that increased overtime up to day 21. Treatment with BN-4, BN-14 and BN-16 to all arthritic rats in this prophylactic model of Freund's Complete Adjuvant induced arthritis was found to be effective and comparable with standard drug used in the study. BN-4 and indomethacin significantly reduced paw volume on 5th day (p<0.05). BN-14 and BN-16 have not shown significant reduction on 5th day when compared with arthritic control group. BN-4 BN-14 and BN-16 compounds displayed significant inhibition in paw volume on 12th day (p<0.01). All three compounds have shown significant inhibition in paw volume on 18th day (p<0.01) when compared with arthritic control. The anti arthritic activity was calculated based on control group and expressed as percent inhibition. The antiarthritic activity of BN4, BN14 and BN16 was comparable (P>0.05) with standard indomethacin group on 12^{th}, 18^{th} and 21^{st} days. In FCA model BN-4, BN-14, BN-16 significantly (p<0.01) reduced the swelling of paws and protected the animals from developing arthritis in prophylactic model. All three compounds show significant inhibition at different time intervals and activity is comparable with standard indomethacin.

Effect of synthetic compounds BN-4, BN-14, and BN-16 on FCA induced arthritis. Table shows paw volume and percentage of inhibition at different time intervals. # Indicates

[symbol]

Significant difference (P<0.05) when compared with Indomethacin group; * indicates no significant statistical difference (p>0.05) when compared with indomethacin group. Results are shown as the Mean±S.E.M. for six animals in each group.

### FCA Induced Model

### Total WBC Count on Termination Day

In arthritic study the total WBC count was measured on termination day of the study for all the groups of rats. White blood cells are major component of the body's immune system. Indications for WBC count include infections and inflammatory diseases (Maria et al., 1983). In arthritic condition there is a mild to moderate increase in WBC count due

| Compound | | % Of | | % Of | | % Of | | % Of |
|---|---|---|---|---|---|---|---|---|
| Code | 5^{th} Day | inhibition | 12^{th} Day | inhibition | 18^{th} Day | inhibition | 21^{st} Day | inhibition |
| Arthritic | 2.38 ± 0.23 | — | 2.48 ± 0.24 | — | 2.62 ± 0.22 | — | 2.37 ± 0.28 | [symbol] |
| Control | | | | | | | | |
| BN-4 | 1.51 ± 0.09 | 36.41 ± 3.84* | 1.42 ± 0.21 | 42.88 ± 8.45* | 1.55 ± 0.20 | 40.71 ± 7.59* | 1.63 ± 0.12 | 31.50 ± 4.8 [symbol] * |
| BN-14 | 1.92 ± 0.03 | 19.47 ± 1.38# | 1.54 ± 0.10 | 37.90 ± 4.12* | 1.51 ± 0.14 | 42.24. ± 5.36* | 1.40 ± 0.02 | 40.93. ± 0.64* |
| BN-16 | 1.81 ± 0.11 | 23.95 ± 4.49# | 1.51 ± 0.10 | 39.11 ± 1.68* | 1.64 ± 0.13 | 37.53 ± 09.04* | 1.59 ± 0.09 | 33.80 ± 01.1 [symbol] * |
| (Indomethacin) | 1.49 ± 0.06 | 37.25 ± 2.36* | 1.35 ± 0.09 | 45.43 ± 3.56* | 1.42 ± 0.02 | 45.80 ± 6.09 | 1.43 ± 0.08 | 39.80 ± 3.3 [symbol] * |

[symbol]

indicates data missing or illegible when filed

to release of IL-1β as inflammatory response. IL-1β increases the production of both granulocyte and macrophages colony stimulating factors. In the present studies the total WBC count was increased in arthritic group of rats where as in test groups of rats the count was significantly (p<0.05; p<0.01) suppressed in both FCA and collagen induced models. The standard drug indomethacin has not shown significant (p>0.05) reduction in WBC count in both models. The development of adjuvant arthritis has been thought to be involved in immunological function, especially the delayed-type hypersensitivity reaction (Swingle, 1974; Chang, 1984; Mohr and Wild.1976). Immunosuppressive agents, cyclophosphamide and azathoprine have been reported to be effective only in a prophylactic regimen for rat adjuvant arthritis (Swingle, 1974; Walz et al., 1971; Pepper et al., 1971)) and immune modulators (gold salts and D-pencillamine) are also active, but their effects depend on the treatment schedule or quantity of drug dose. However the immunolomodulatory studies for synthetic compounds have not performed. The total

WBC count for different groups as follows Normal control (13.03±3.25), Arthritic control (25.90±1.30), BN-4 (18.53±1.36), BN-14, (19.70±0.66), BN-16 (17.3±1.63) and Indomethacin (20.67±0.59). In collagen induced study the total WBC count was analyzed by using cell counter and count as follows; Normal control (11.95±2.26), Arthritic control (18.88±0.53), BN-4 (13.57±0.63), BN-14, (14.88±2.64), BN-16 (15.70±1.28) and Indomethacin (19.70±2.23).

### Effects of Thienopyridine Derivatives on Expression of TNF-α and IL1-β in Rat Paw Tissue —FCA Model.

Proinflammatory cytokines TNF-α and IL-1β are shown to play an important role in pathophysiology of arthritis development in animal models and humans. (ref).The effects of these cytokines in arthritic joints appear to be multiple and include the expression of adhesion and chemo attractant molecules and facilitation of leukocytes influx and activation.

In FCA induced study paw tissues were collected on termination day and estimated TNF-α and IL-1β levels for all the groups by ELISA method. In the arthritic group of rats cytokine levels were increased significantly when compared with normal control group. Measurement of cytokines in paw tissues revealed that the compounds BN-4, BN-14, and BN-16 show significant (P<0.05) inhibition in IL-1 TNF-α levels compared with arthritic control. TNF-α in Paw Tissue was suppressed more significantly (p<0.01) than IL-1 Beta.

Inflammatory process in CIA rats treated with vehicle shown substantial increase in systemic levels of pro-inflammatory cytokines TNF-α and IL-1β; while the Thienopyridine derivatives BN-4, BN-14 and BN-16 suppressed the levels at the dose of 100 mg/kg /day in prophylactic treatment. It is believed that these proinflammatory cytokines help to propagate a local or systemic inflammatory process, and to induce biosynthesis and secretion of metalloproteinases (MMPs) and osteoclasts that critically contribute to degradation of extracellular matrix and erosion of bone respectively (Burger et al., 1998; Gravallese and Goldring, 2000; Smolen and Steiner, 2003). In Collagen induced study the cytokine levels were determined on 24 the day of the study and the compounds were significantly reduced the levels of IL-1 Beta comparing with vehicle control. In paw tissues the compounds BN-4, BN-14 has shown more significant inhibition (p<0.01) in both TNF-α and IL-1β in paw tissues where as BN-16 and indomethacin shown moderate inhibition (p<0.05).

### TNF-α and IL1-β in Rat Paw Tissue

### FCA Model

| | TNF-α | | IL-1 Beta | |
|---|---|---|---|---|
| | Concentration | | Concentration | |
| Compound | (Pg/gm of | | (ng/gm of | |
| Code | tissue) | % Inhibition | tissue | % Inhibition |
| Normal | 12.14 ± 3.75 | − | 39.93 ± 6.80 | − |
| Control | | | | |
| Arthritic | 396.9 ± 4.83 | − | 127.92 ± 18.24 | − |
| Control | | | | |
| BN-4 | 226.58 ± 15.07 | 42.9 ± 3.78 | 92.07 ± 10.70 | 33.59 ± 5.53 |
| BN-14 | 244.6 ± 22.3 | 38.03 ± 9.73 | 113.0 ± 26.8 | 38.49 ± 14.97 |
| BN-16 | 230.49 ± 6.27 | 37.42 ± 5.57 | 68.87 ± 21.1 | 40.51 ± 9.56 |
| Indo- | 264.49 ± 12.08 | 29.06 ± 0.06 | 81.02 ± 17.98 | 40.42 ± 8.73 |
| methacin | | | | |

### Cytokine Estimation in Serum

On termination day blood samples were collected and serum was used for the estimation of Both TNF-α and IL-1β levels in FCA and Collagen induced studies. Both the parameters were determined using ELISA kits. TNF-α could not be determined as their expression is very less in serum samples of the rats in the two models studied, where as IL-1β expression in serum was good. Daily treatment of synthetic compounds by oral administration was effective in reducing the serum IL-1β levels (P<0.01) when compared with arthritic control group. In collagen study the compounds BN-4 and BN-16 more significantly (p<0.01) reduced the levels in serum and BN-14, Indomethacin have shown moderate inhibition (p<0.05) when compared with arthritic control group.

### IL1-β in Rat Serum —FCA Induced Arthritis

| | IL-1 Beta concentration | |
|---|---|---|
| Compound Code | (Pg/ml of tissue) | Percentage of Inhibition |
| Normal Control | 6.16 ± 2.10 | − |
| Arthritic Control | 113.0 ± 5.67 | − |
| BN-4 | 22.83 ± 9.93 | 29.28 ± 3.60 |
| BN-14 | 50.83 ± 6.11 | 22.17 ± 6.51 |
| BN-16 | 17.0 ± 4.50 | 32.48 ± 6.22 |
| Indomethacin | 46.0 ± 7.91 | 20.04 ± 5.33 |

### Adenosine Antagonistic Activity

Thienopyridines were proven as potent cytotoxic and anti-inflammatory agents in the previous experimental results. In cytotoxic MTT assay, activity of these molecules may be due to the involvement of adenosine receptors. Further, to find the bronchodilatory activity of these moieties and to find the involvement of adenosine receptors in their action, non-invasive and invasive adenosine mediated bronchoconstriction and lung inflammation in an allergic mouse model was performed.

Male mice, 6-8 weeks of age, free of specific pathogens were obtained from National Institute of Nutrition. The animals were maintained on a ragweed-free diet. All experimental protocols used in this study were under a protocol approved by animal ethical committee. Sensitization was performed according to the modified procedure described by Fan et al, 2002. Mice were sensitized on days 1 and 6 with i.p. injections of ragweed allergen (200 μg/200 μl of water for injection). Non-sensitized control animals received only water for injection with the same volumes. Ten days after sensitization, the mice were placed in plexiglas chamber and challenged with 0.5% ragweed or with 0.9% saline as a control for twenty minutes both in the morning and evening for three days. Then mice were divided into different groups consisting of six animals each. The groups including a control without sensitization; a sensitized group and a standard group along with groups of the test molecules were separated. Twenty-four hours after last challenge with ragweed, the mice were given an oral dose of test molecules and theophylline (100 mg/kg body weight) as acacia suspension. Two hours after administration of the oral dose, the mice were challenged with adenosine solution (24 mg/ml). The variation in respiration was recorded after anaesthetizing them with pentobarbitone sodium (0.1 ml 200 mg/ml ip) using kymograph. Then, mice were sacrificed and trachea was cannulated to collect the bronchoalveolar lavage fluid. This was performed by pumping 1 ml phosphate buffered saline into lungs through tracheal cannula and collecting the sample. Analysis of BAL fluid was performed to get the cellular differentials. The BAL fluid was centrifugated at 1500 rpm for six minutes at room temparature. After removing the supernatant, BAL cells were resuspended in 1 ml of phosphate buffered saline. Total cells were then counted manually in a hemocytometer chamber. A differential count of at least 300 cells was made according to the standard morphologic criteria and results were mentioned in table.

### Antidiabetic Activity

Chemicals: α-Glucosidase [(EC 3.2.1.20) Type I: from bakers yeast], rat intestinal acetone powder (as a source of crude intestinal α-glucosidase), α-amylase [EC 3.2.1.1) Type VI B: from porcine pancreas], p-nitrophenyl α-D-glucopyranoside, 3,5-dinitro salicylic acid, soluble potato starch and 1-deoxyrojirimycine, were purchased from Sigma Chemical Co., St Louis, Mo., USA. Other chemicals of analytical grade were procured from indigenous manufacturers.

Experimental Animals—Wistar rats of either sex were used for the present study. Animals selected for use in the study were of uniform age and body weight. A minimum of 40 rats, from which eventually 20 rats were selected. Their body weights were in the range of 150-180 gm. The health status of the animals was ascertained by a veterinary surgeon. Animals were divided in to Control, Test and Standard (Acarbose) groups each containing 6 rats. Animals were acclimatized for at least two weeks prior to start of the study. The animals were provided with pellet diet manufactured by NIN, HYDERABAD, India and drinking water adlibitum.

Statistical Analysis: Results of in vivo experiments were evaluated by performing one-way

| | Broncho- | Total | Differential count (%) | | |
|---|---|---|---|---|---|
| Compd | dilation | count | Monocytes | Lymphocytes | Eosinophils [symbol] |
| BN-1 | + | 12 | 20 | 65 | 12 |
| BN-2 | Not | − | − | − | − |
| | performed | | | | |
| BN-3 | + | 14.5 | 15 | 75 | 14 |
| BN-4 | + | 9 | 20 | 80 | − |
| BN-5 | Not | − | − | − | − |
| | performed | | | | |
| BN-6 | + | 10.5 | 10 | 75 | 15 |
| BN-7 | + | 7 | 10 | 70 | 20 |
| BN-8 | + | 14 | 10 | 70 | 20 |
| BN-13 | + | 8 | 16.5 | 74 | 20 |
| BN-14 | + | 13.5 | 13.5 | 80 | 15 |
| BN-15 | + | 13 | 16.5 | 63.5 | 10 |
| BN-16 | + | 8 | 15 | 90 | 12 |
| BN-17 | + | 9 | 9 | 75 | 25 |
| BN-18 | − | 18 | 15 | 70 | 20 |
| BN-19 | + | 11.5 | 10 | 80 | 10 |
| Control | | 7 | 10 | 60 | 5 |
| Sensitized | | 13.5 | 25 | 95 | 20 |
| [symbol] | | | | | |

[symbol]

indicates data missing or illegible when filed

ANNOVA followed by Dunnet's multiple comparison procedure. Significance was evaluated at p value of 0.05.

Compound Preparation The required quantity of the synthetic compounds were weighed and made suspension with gum acacia (2%). The test compounds were administered to all rats by per oral route. Acarbose suspension was also prepared in gum acasia suspension and given by oral route.

In-vitro a-Glucosidase Inhibition Assay: Yeast and intestinal a-glucosidase inhibitory activities were determined as per reported methods (Babu et al., 2004; Mc Dougall et al., 2005). Rat intestinal acetone powder in normal saline (100% w/v) was sonicated properly and the supernatant was used as a source of crude intestinal a-glucosidase after centrifugation. In brief, 20 μl, of test samples (100 mg/ml DMSO solution) were reconstituted in 100 μl of 100 mM-Phosphate buffer (pH 6.8) in a 96-well microplate and incubated with 50 μl yeast a-glucosidase (0.76 μ/ml in same buffer) or crude intestinal a-glucosidase for 5 min before 50-μl substrate (5 mM, p-nitrophenyl-a-D-glucosidase prepared in same buffer) was added. Release of p-nitrophenyol was measured at 405 nm spectrophotometrically (Spectra Mas Plus 384, Molecular Device corporation, Sunnyvale, Calif., USA) 5 min after incubation with substrate, individual blank for test samples were prepared to correct background absorbance where the substrate was replaced with 50 μl of buffer. The control sample contained 20 μl DMSO in place of test samples. The percentage of enzyme inhibition was calculated as (A−B/A)×100 where A is represents the absorbance of the control without test samples, and B represents the absorbance in the presence of test samples. At least (10 mg/ml) DMSO was considered to study the concentration dependent enzyme inhibition in the case of the intestinal a-glucosidase assay and calculation of IC50 values (Concentration required to inhibit 50% enzyme activity); all the tests were run in duplicate. The IC50 values were calculated by applying logarithmic regression analysis from the mean inhibitory values.

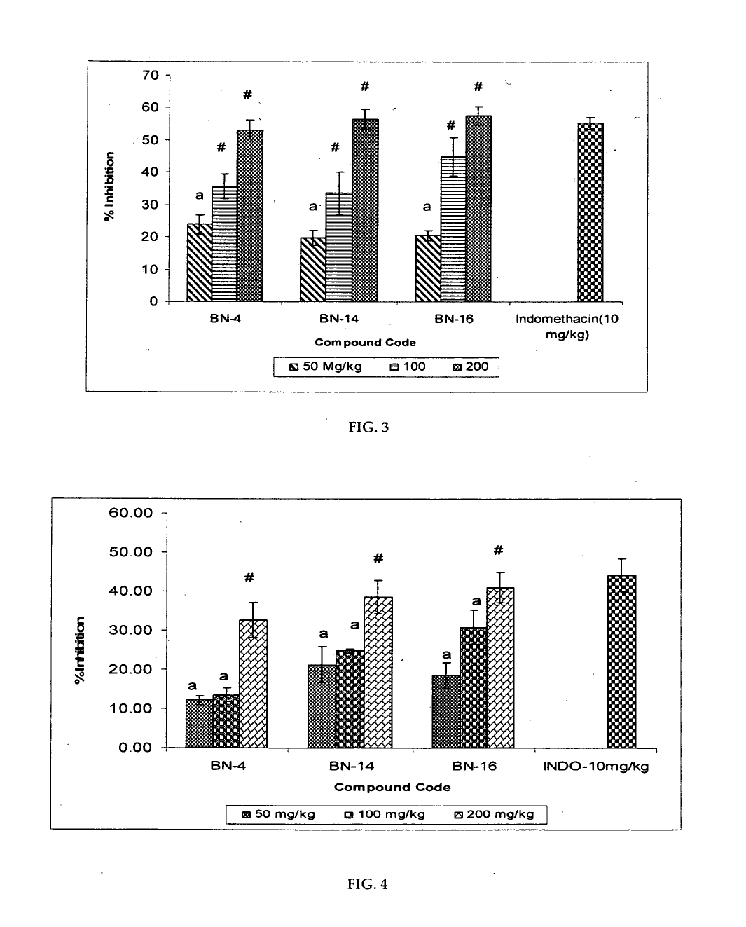

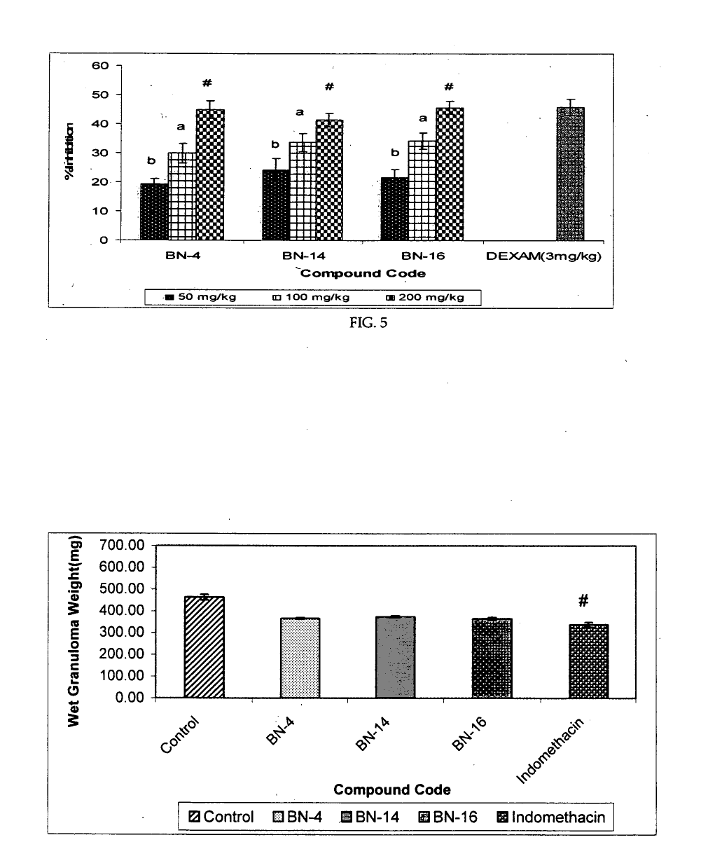

Both yeast and mammalian (rat intestinal) α-glucosidase models were used for the screening of these compounds for antidiabetic activity. Two compounds, BN-2 and BN-14 of this series have shown strong significant inhibitory activity (p>0.05; p>0.01) for yeast α-glucosidase enzyme inhibitory activity in a dose dependent manner (Table-6 and FIGS. 1 & 2). They are comparable with standard Acarbose at the dose of 1.25 and 5 mg/ml respectively. Five of them, BN-5, BN-6, BN-13, BN-15, BN18 and BN-19 have shown (p<0.05) moderate inhibitory activity on yeast α-glucosidase enzyme. On the mammalian α-glucosidase the following compounds, BN-13, BN-14, BN-15 and BN-17 have shown significant inhibitory activity p<0.01 (FIG. 3 & FIG. 4) Five of them, BN-2, BN-3, BN-4, BN-7 and BN-18 have shown moderate inhibitory activity on mammalian α-glucosidase enzyme. The compounds, BN-13, BN-14, BN-15 and BN-17 at different concentration 5, 2.5, 1.25, 0.62, 0.31, 0.15, 0.07, 0.03 mg/ml have shown a concentration-dependendent reduction in Alfa glucosidase inhibition assay. Thus the highest concentration 5 mg/ml tested have shown maximum inhibition effect 76.13%, 58.37%, 88.87%, 75.39%.

### Invitro α-glucosidase Inhibition Assay

| | Yeast α Glucosidase | Mammalian |
|---|---|---|
| Compound | Inhibition | α Glucosidase Inhibition |
| Code | (Mean ± S.E) | (Mean ± S.E) |
| BN-1 | 0.00 ± 0.0 | 0.0 ± 0.0 |
| BN-2 | 56.74 ± 0.28 | 40.0 ± 2.5 |
| BN-3 | 20.41 ± 1.09 | 46.07 ± 0.9 |
| BN-4 | 18.16 ± 1.40 | 38.37 ± 1.0 |
| BN-5 | 41.80 ± 0.94 | 0.0 ± 0.0 |
| BN-6 | 37.62 ± 0.02 | 19.92 ± 1.27 |
| BN-7 | 0.0 ± 0.0 | 50.19 ± 0.93 |
| BN-8 | 27.75 ± 0.19 | 10.76 ± 0.92 |
| BN-13 | 37.41 ± 0.19 | 76.13 ± 0.03 |
| BN-14 | 72.25 ± 1.87 | 58.37 ± 2.2 |
| BN-15 | 37.01 ± 2.10 | 88.87 ± 1.35 |
| BN-16 | 24.12 ± 0.10 | 4.46 ± 1.77 |
| BN-17 | 9.40 ± 4.49 | 75.39 ± 4.15 |
| BN-18 | 46.22 ± 2.58 | 46.22 ± 4.15 |
| BN-19 | 47.08 ± 1.72 | 0.0 ± 0.0 |
| Acarbose | 78.59 ± 1.13 | 88.5 ± 2.03 |
| [symbol] | | |

[symbol]

indicates data missing or illegible when filed

### Mammalian α-Glucosidase Inhibitory Activity

### Dose Response Study

| Concentration | Compound Code (Mean ± S.E) | | | | |
|---|---|---|---|---|---|
| (mg/ml | BN-13 | BN-14 | BN-15 | BN-17 | Acarbose |
| 5 | 76.13 ± 0.03 | 58.37 ± 2.20 | 88.87 ± 1.35 | 75.39 ± 13.76 | 88.55 ± 3.21 |
| 2.5 | 71.39 ± 1.82 | 54.74 ± 1.01 | 83.34 ± 3.13 | 70.69 ± 2.75 | 78.59 ± 2.78 |
| 1.25 | 67.24 ± 1.08 | 51.56 ± 0.74 | 78.49 ± 1.88 | 66.57 ± 1.11 | 64.28 ± 3.25 |
| 0.625 | 52.97 ± 1.47 | 40.62 ± 0.63 | 61.83 ± 0.26 | 52.44 ± 1.56 | 55.18 ± 4.12 |
| 0.3125 | 44.87 ± 1.62 | 34.49 ± 1.19 | 52.37 ± 1.64 | 44.41 ± 1.52 | 49.28 ± 2.36 |
| 0.15625 | 34.89 ± 2.28 | 26.76 ± 1.37 | 40.72 ± 2.26 | 34.53 ± 2.61 | 40.87 ± 1.52 |
| 0.078125 | 27.07 ± 1.93 | 20.75 ± 2.19 | 31.57 ± 2.74 | 26.77 ± 1.83 | 31.37 ± 2.56 |
| 0.039063 | 10.06 ± 1.36 | 7.79 ± 3.11 | 11.85 ± 3.45 | 10.04 ± 4.42 | 12.82 ± 3.14 |
| IC₅₀ | 0.509 | 1.56 | 0.299 | 0.528 | 0.372 |

Table shows dose response study of Thienopyridine derivatives on Mammalian a glucosidase inhibition assay. Values are mentioned as Mean±S.E

### Yeast Alfa Glucosidase Inhibition Assay

### Dose Response Study

| Concentration | % Inhibition (Mean ± S.E) | | |
|---|---|---|---|
| (mg/m1) | BN-2 | BN-14 | Acarbose |
| 5 | 56.54 ± 0.28 | 72.25 ± 1.87 | 78.59 ± 1.13 |
| 2.5 | 53.02 ± 1.13 | 67.76 ± 1.18 | 64.28 ± 0.16 |
| 1.25 | 43.93 ± 1.92 | 63.82 ± 1.71 | 55.18 ± 0.41 |
| 0.625 | 39.17 ± 0.87 | 50.27 ± 3.39 | 49.28 ± 0.15 |
| 0.3125 | 33.38 ± 0.76 | 42.844 ± 1.38 | 40.87 ± 2.18 |
| 0.15625 | 25.90 ± 0.44 | 33.31 ± 0.04 | 31.37 ± 0.40 |
| 0.078125 | 20.13 ± 1.37 | 25.84 ± 0.13 | 12.82 ± 0.43 |
| 0.039063 | 15.30 ± 2.69 | 19.65 ± 0.00 | 7.05 ± 0.00 |
| IC50 | 1.905 | 0.569 | 0.744 |

Table shows dose response study of Thienopyridine derivatives on Yeast a glucosidase inhibition assay. Values are mentioned as Mean±S.E

a-Amylase Inhibition Assay: The assay method was adopted from Ali et al. (2006) and modified accordingly to suite microplate reading. In brief, 40 μL of test samples (10 mg/mL DMSO) were reconstituted in 160 μL of 20 mM phosphate buffer (pH 6.9) containing 6.7 mM sodium chloride in 2 mL Eppendorf tubes and incubated with 200 μL of porcine pancreatic α-amylase) (4 U/mL prepared in ice cold distilled water) for 5 min. The reaction was started by the addition of 400-μL soluble potato starch solution (0.5% w/v in 20 mM phosphate buffer, pH 6.9). Exactly 3 min after incubation, 400 μL DNS color reagent (1.0 g of 3,5-dinitrosalicylic acid, 30 g of sodium potassium tartrate and 20 mL of 2 N NaOH to a final volume of 100 mL in distilled water) was added. Closed tubes were placed in a water bath (85-90° C.) for 10 min to develop color and cooled thereafter. 50 μL of reaction mixtures was diluted with 175 μL of distilled water in a 96-well microplate. α-Amylase activity was determined by measuring the absorbance of the mixture at 540 nm spectrophotometrically as above. Individual blanks were prepared to correct for the background absorbance due to test samples. In this case DNS solution was added in tubes before addition of substrate. The percentage inhibition of enzyme activity was calculated as above. All the tests were run in duplicate in this assay.

Thienopyridine derivatives were screened for porcine α-amylase inhibitory activity. Some compounds showed strong inhibitory activity in this model. The amount of maltose released during the reaction for all the compounds was determined by using maltose standard graph. The percentage inhibition of the test compounds was calculated based on the maltose concentration of the individual compounds. Alpha amylase catalyses the hydrolysis of alpha-1,4-glucosidic linkage in starch and related polysaccharides, have also been target for suppression of post-prandial hyperglycemia (PPHG). In this series of compounds BN-8, BN-17, BN-15, BN-13 and BN-2 have shown strong inhibitory activity on alpha amylase. The compounds BN-2, BN-6 and BN-16 have shown mild inhibition in Alfa amylase. Percentage amylase inhibition was calculated based on the amount of maltose released during the reaction. The compounds BN-8, BN-17, BN-15, BN-13 liberated significant low amount of maltose concentration when compared with their respective control p<0.01. Significant decrease in maltose release is the good index for the amylase enzyme inhibition, which is responsible for digestion of carbohydrates. The compounds which are having both alpha amylase as well as alpha glucosidase inhibitor activity they can be more useful as anti-diabetic agents as they are inhibiting both the primary enzymes which are involved in the digestion of the carbohydrates in the gut.

| Compound Code | Maltose released (mg/ml) | % Inhibition |
|---|---|---|
| Control-1 | 2.147 | − |
| Control-2 | 3.429 | |
| BN-1 b | 2.20 | −3.12 |
| BN-2 b | 2.095 | 39.71631 |
| BN-4 b | 3.496 | −2.00946 |
| BN-5 b | 3.226 | 4.846336 |
| BN-6 b | 2.548 | 26.24113 |
| BN-7 a | 2.163 | −0.76482 |
| BN-8 a | 0.913 | 59.46463 |
| BN-13 a | 1.278 | 41.8738 |
| BN-14 a | 2.210 | −3.05927 |
| BN-15 a | 1.179 | 46.65392 |
| BN-16 a | 1.492 | 31.54876 |
| BN-17 a | 1.028 | 53.91969 |
| BN-18 b | 3.972 | −16.1939 |
| BN-19 b | 3.282 | 4.373522 |

Table shows percentage of α-amylase inhibition of the Thienopyridine derivatives at the concentration of 10 mg per ml in in-vitro study.

Antidiabetic activity by Starch model: The method of Rao et al., 2009;Tiwari et al was employed. Animals were fasted over night (12 hrs). Basal blood glucose levels were estimated for all the animals prior to dosing in test and standard groups. This was designated as “0” min reading. The test compounds and Acarbose were administered orally to the rats in test and standard groups at doses of 100 mg/k/kg and 10-mg/kg-body weight respectively. After test compounds administration animals in all three groups received Starch aqueous solution (2 gm/kg body weight). Blood glucose levels were monitored at 30, 60, 90 and 120 min after the administration of Starch in all the three groups. The percentage of increase in, blood glucose levels was calculated with respect to the control group at each time point. The results were presented in tables.

On the basis of the results of the in vitro experiments four compounds were selected for the in vivo anti diabetic activity on the starch loaded model (BN-13, BN-14, BN-15, and BN-17). These compounds have shown good activity for both mammalian alpha glucosidase as well as alpha amylase inhibitory activity in in-vitro models. Blood glucose levels were estimated every 30 min and compared with diabetic control as shown in the Table. Percent increase in glucose concentration at each time point calculated with respective control. Oral administration of all four test compounds at the dose of 100 mg/kg have shown the significant reduction (p<0.01) in percentage increase in blood glucose levels in all time intervals compared to diabetic control. BN-13 showed greatest inhibitory activity and it is almost comparable to standard drug acarbose at the dose of 10 mg/kg among all the compounds studied. Acarbose like drugs that inhibit α-glucosidase present in the epithelium of the small intestine, have been demonstrated to decrease post-prandial hyperglycaemia (Sigma & Chakrabarthi, 2004) and improve impaired glucose metabolism without promoting insulin secretion in NIDDM patients (Carrascosa et al., 2001) Thus these compounds can serve as lead compounds in the search for the drugs to treat postprandial hyperglycemia. These medications are most useful for people who have just been diagnosed with type 2 diabetes and who have blood glucose levels only slightly above the level considered serious for diabetes. These drugs are also useful for the people who are taking Sulfonylurea medication or Metformin, who need an additional treatment to control their blood glucose levels within a safe range. Since the compounds were able to retard and delay the absorption of carbohydrate in starch loaded study Thienopyridine derivatives seems to be promising in the treatment of type2 diabetes mellitus by reducing postprandial hyperglycaemia in animal models.

### Anti-Diabetic Activity

### Starch Loaded Model

| | 0 | 30 | % Increase | 60 | % Increase | 90 | % Increase | 120 | % Increase |
|---|---|---|---|---|---|---|---|---|---|
| Group | min | min | in glucose | min | in glucose | min | in glucose | min | in glucose |
| CONTROL | 83.72 ± | 151.47 ± | 81.13 ± | 146.6 ± | 75.28 ± | 133.9 ± | 60.20 ± | 126.18 ± | 50.75 ± |
| | 1.29 | 3.42 | 4.80 | 3.54 | 4.73 | 1.63 | 2.99 | 3.11 | 3.11 |
| BN-13 | 81.67 ± | 108.37 ± | 32.75 ± | 110.82 ± | 35.89 ± | 106.50 ± | 30.53 ± | 106.30 ± | 30.27 ± |
| | 1.36 | 2.55 | 2.75 | 2.62 | 3.90 | 2.95 | 3.74 | 3.22 | 4.00 |
| BN-14 | 75.5 ± | 103.83 ± | 37.65 ± | 102.80 ± | 36.02 ± | 109.33 ± | 45.00 ± | 99.83 ± | 32.21 ± |
| | 1.12 | 6.53 | 8.85 | 5.37 | 6.16 | 4.02 | 5.93 | 5.61 | 6.88 |
| BN-15 | 81.45 ± | 115.50 ± | 42.13 ± | 123.03 ± | 51.64 ± | 113.75 ± | 39.76 ± | 116.05 ± | 42.80 ± |
| | 2.16 | 1.98 | 3.22 | 2.76 | 5.60 | 3.19 | 2.80 | 3.16 | 4.34 |
| BN-17 | 82.40 ± | 124.60 ± | 51.74 ± | 125.13 ± | 52.35 ± | 123.13 ± | 49.67 ± | 122.80 ± | 49.64 ± |
| | 2.38 | 2.47 | 4.75 | 2.55 | 4.54 | 2.41 | 2.00 | 1.18 | 4.55 |
| ACARBOSE | 83.50 ± | 103.17 ± | 23.55 ± | 110.17 ± | 32.03 ± | 109.83 ± | 31.78 ± | 108.62 ± | 30.27 ± |
| | 1.12 | 3.72 | 3.42 | 2.66 | 2.46 | 2.30 | 3.31 | 2.57 | 3.22 |

1. Thienopyridine compounds of the formula I below 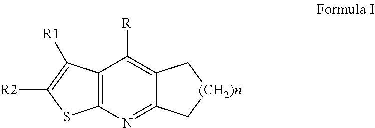
where n is 1, 2, 3 or 4,
R1 and R2 are independently selected from the group consisting of , alkyl excluding CH₃ and aryl; wherein, when R1 is aryl R2 is not aryl and when R2 is aryl R1 is not aryl; or R1+R2 is selected from cyclopentyl, cycloheptyl, bicycloalkyl, and alkyl of more than 2 carbon chains;
R is selected from the group consisting of amine, sulfonamide, sulfonyl alkyl, alkyl or cycloalkyl, aryl, hydroxamate, and heterocyclic moieties;
the said heterocyclic moiety is selected from imidazole, triazole, tetrazole, pyridine, benzimidazole, quinazoline, quinoline, thiophene, thienopyrimidine, thienopyridine, acridine, indole, pyrrole and benzofuran and pharmaceutically acceptable salts thereof.

2. A thienopyridine compound as claimed in claim 1 wherein the ring is a substituted ring wherein the substituents are selected from the group consisting of —H, —(C₁-C₃) alkyl, —O(C₁-C₃) alkyl, —F, —CF₃, —NH₂, —N(CH₃), —N(CH₃)₂, —SH, —SCH₃, —SCH₂CH₃ and any combination thereof.

3. A compound as claimed in claim 1 selected isomers of compounds of the general formula I, inclusive of their stereoisomeric forms, polymorphs, acid addition salts, base addition salts, and prodrugs thereof.

4. A compound as claimed in any preceding claim wherein the acid addition salt is selected from the group consisting of acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, napthelene-2-benzoate, isobutyrate, phenylbutyrate, b-hydroxybutyrate, butyne-1-4-dioate, hexyne-1-4-dioate, caprate, caprylate, cinnamate, citrate, formate, fumerate, glycollate, heptanoate, hippurate, lactate, maleate, malate, hydroxymaleate, malonate, madelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, ethanesulfonate, naphthalene-1-sulfonate, naphthalene-2-sulfonate, p-toluenesulfonate, xylenesulfonate, and tartarate.

5. A compound as claimed in any preceding claim wherein the acid addition salt is selected from the group consisting of hydrochloride, hydrobromide, citrate and oxalate.

6. A compound as claimed in claims 1 to 3 wherein the basic addition salts are formed from inorganic bases selected from the group consisting of sodium, potassium, lithium, calcium, aluminium, ammonium, barium, zinc, and magnesium.

7. A compound as claimed in claims 1 to 3 wherein the basic addition salt is selected from the group consisting salts obtained from an organic base selected in turn from the group consisting of N—N′-dibenzylethelynediamine, choline, diethanolamine, ethelenediamine, N-methylglucamine, triethylamine, dimethylamine and procaine.

8. A compound as claimed in claims 1 to 3 wherein the salt is an amine salt such as an arginate.

9. A compound as claimed in claims 1 to 3 wherein the prodrug is selected from the group obtained by conjugation of compounds of formula I with sugar moieties with suitable spacers, and alkyl esters obtained by reaction of the parent acid with a suitable alcohol, or amides obtained by reaction of parent acidic compound with a suitable amine.

10. A compound as claimed in any preceding claim wherein aryl is selected from phenyl, biphenyl, benzyl, naphthyl, anthryl, phenanthryl, fluorenyl and indenyl.

11. A compound as claimed in any of claims 1 to 10 wherein the heterocycle is selected from the group consisting of imidazole, triazole, tetrazole, indole, and pyrrole.

12. A compound as claimed in claim 11, wherein the heterocycle has one or more heteroatoms selected from O, S and N in the aromatic ring.

13. A compound as claimed in claim 1 wherein the compound is 2,3,6,7,8,9-hexahydro-1H-benzo[4,5]thieno[2,3-b]cyclopenta[e]pyridin-10-amine.

14. A compound as claimed in claim 1 wherein the compound is 2,3,4,7,8,9,10,11-octahydro-1H-benzo[4,5]thieno[2,3-b]cyclohepta [e]pyridin-12-amine.

15. A compound as claimed in claim 1 wherein the compound is 1,2,3,6,7,8-hexahydrocyclopenta[b]cyclopenta[4,5]thieno[3,2-e]pyridin-9-amine.

16. A compound as claimed in claim 1 wherein the compound is 2,3,6,7,8,9-hexahydro-1H-cyclopenta[4,5]thieno[2,3-b]quinolin-10-amine.

17. A compound as claimed in claim 1 wherein the compound is 7-methyl-2,3,6,7,8,9-hexahydro-1H-cyclopenta[4,5]thieno[2,3-b]quinolin-10-amine.

18. A compound as claimed in claim 1 wherein the compound is 1,2,3,6,7,8,9,10-octahydrocyclohepta[b]cyclopenta[4,5]thieno[3,2-e]pyridine-11-amine.

19. A compound as claimed in claim 1 wherein the compound is 2,3,6,7,8,9,10,11-octahydro-1H-cycloocta[b]cyclopenta[4,5]thieno[3,2-e]pyridin-12-amine.

20. A compound as claimed in claim 1 wherein the compound is 1,2,3,6,7,8,9,10-octahydrocyclohepta[4,5]thieno[2,3-b]cyclopenta[e]pyridin-11-amine.

21. A compound as claimed in claim 1 wherein the compound is 2,3,4,7,8,9,10,11-octahydro-1H-cyclohepta[4,5]thieno[2,3-b]quinolin-12-amine.

22. A compound as claimed in claim 1 wherein both R1+R2 is alkyl.

23. A compound as claimed in claim 1 wherein R1+R2 is selected from the group consisting of , —(CH₂)₃—, —(CH₂)₅—.

24. A compound as claimed in claims 1 and 28 wherein n is 1, 2 or 3.

25. A compound as claimed in claims 1 and 30 wherein ,n is 1, 2 or 3.

26. A compound as claimed in claims 1 to 30 wherein R is NH₂.

27. A method for the preparation of a compound of general formula I, said method comprising
(a) synthesizing 2-amino 3-cyano thiophene;
(b) reacting the 2-amino 3-cyano thiophene with a cyclic ketone under conditions suitable to obtain the corresponding product of formula I 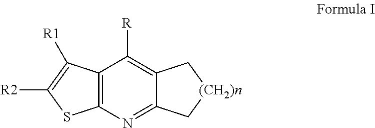
where n is 1, 2, 3 or 4
R1 and R2 are independently selected from the group consisting of alkyl excluding CH₃ and aryl; wherein, when R1 is aryl R2 is not aryl and when R2 is aryl R1 is not aryl; or R1+R2 is selected from cyclopentyl, cycloheptyl, bicycloalkyl, and alkyl;
R is selected from the group consisting of amine, sulfonamide, sulfonyl alkyl, alkyl or cycloalkyl, aryl, hydroxamate, and heterocyclic moieties;
wherein the heterocyclic moiety is selected from imidazole, triazole, tetrazole, pyridine, benzimidazole, quinazoline, quinoline, thiophene, thienopyrimidine, thienopyridine, acridine, pyrrole, indole and benzofuran and pharmaceutically acceptable salts thereof.

28. A method as claimed in claim 34 wherein the reaction with cyclic ketone is carried out by formation of a zinc chloride complex, followed by treatment with a base to precipitate the product from the complex, followed by separation and purification, and if desired, conversion to the desired ester, or salt form.

29. A method as claimed in claims 34 and 35 wherein the reaction with zinc chloride is carried out by heating under reflex for a time suitable to form a complex.

30. A method as claimed in claims 34 to 36 wherein the thiophene and the cyclic ketone are reacted at a molar ratio of 1:2.

31. A method as claimed in any of claims 34 to 37 wherein the base is NaOH.

32. A method as claimed in any of claims 34 to 38 wherein the 2-amino, 3-cyano thiophene is prepared by reacting sulphur, melanonitrile and respective ketone in the presence of an alcohol under stirring.

33. A method as claimed in any of claims 34 to 39 wherein the compound of formula I is reacted with an equimolar amount or an excess of acid in a neat or in a suitable inert solvent to form the corresponding acid addition salt.

34. A method as claimed in claim 40 wherein the acid is selected from the group consisting of hydrochloric, hydrobromic, hydroiodic, nitric, sulfuric, phosphoric, hypophosphoric, aliphatic mono and dicarboxylic acid, phenyl substituted alkanoic acids, hydroxyalkanoic and hydroxyalkandioic acids, aromatic acids, aliphatic and aromatic sulfonic acids.

35. A method as claimed in any of claims 34 to 39 wherein the compound of formula I is reacted in an inert suitable solvent or a neat solvent with an equimolar or excess amount of a base to form the corresponding base addition salt.

36. A method as claimed in claim 42 wherein the base is selected from sodium, potassium, lithium, calcium, aluminium, ammonium, barium, zinc, magnesium, N—N′-dibenzylethelynediamine, choline, diethanolamine, ethelenediamine, N-methylglucamine, triethylamine, dimethylamine, and procaine, and amino acids to obtain the respective basic addition salts.

37. A method as claimed in any of claims 34 to 41 wherein the salt is formed by sending the dry acidic gas into the methanolic solution of the compound.

38. A method as claimed in any of claims 34 to 39 wherein the prodrug is obtained by conjugation of compounds of formula (I) with sugar moieties adding suitable spacers, or alkyl esters prepared by the reaction of the parent acidic compound with a suitable alcohol, or amides prepared by reaction of the parent acid compound with suitable amine.

39. A pharmaceutical composition comprising the compound of formula I or a salt or ester or prodrug form thereof with one or more pharmaceutically acceptable excipients.

40. A pharmaceutical composition comprising the compound of formula I 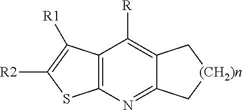
where n is 1, 2, 3 or 4,
R1+R2 are independently selected from the group consisting of alkyl excluding CH₃ and aryl; wherein, when R1 is aryl R2 is not aryl and when R2 is aryl R1 is not aryl; or R1+R2 is selected from cyclopentyl, cycloheptyl, bicycloalkyl, and alkyl of more than 2 carbon chains;
R is selected from the group consisting of amine sulfonamide, sulfonyl alkyl, alkyl or cycloalkyl, aryl, hydroxamate, and heterocyclic moieties;
wherein the said heterocyclic moiety is selected from imidazole, triazole, tetrazole, pyridine, benzimidazole, quinazoline, quinoline, thiophene, thienopyrimidine, thienopyridine, acridine, indole, pyrrole and benzofuran and pharmaceutically acceptable salts thereof
with a conventional active agent for treatment of diabetes, cancer, arthritis or inflammation.

41. A composition as claimed in claim 47, wherein het additional active selected from the group consisting of alkylating agents, antimetabolites, antibiotics, immunomodulating agents, nucleotide derivatives, cyclin dependent kinase inhibitors, interferon like agents and histone deacytalase inhibitors.

42. A composition as claimed in claim 47 wherein the additional active is selected from the group consisting of COX-II inhibitors such as nimuselide, celocoxib, etorocoxib, and valdicoxib.

43. A composition as claimed in claim 47 wherein the additional active .is selected from the group consisting of sulphonylureas, Biguanides, Meglitinides, Glitazones, and -Glucosidase Inhibitors.

44. A composition as claimed in any of claims 46 to 50 wherein the pharmaceutically acceptable excipients are selected from the group consisting of pharmaceutically acceptable carrier or diluent.

45. A composition as claimed in claim 51 wherein the carrier or diluents is selected from the group consisting of water, salt solutions, alcohols, polyethylene glycols, polyhydroxy ethoxylated castor oil, peanut oil, olive oil, gelatine, lactose, sucrose, cyclodextrin, amylose, magnesium stereate, talc, agar, silicic acid, fatty acids, fatty acid amines, fatty acid monoglycerides, fatty acid diglycerides, polyoxyethylene, hydroxymethylcellulose, and polyvinylpyrrolidine.
